(19) United States
(12) Patent Application Publication
(10) Pub. No.: US 2023/0183321 A1
(43) Pub. Date: Jun. 15, 2023

(54) RCOM PROTEIN BASED CARBON MONOXIDE SCAVENGERS AND PREPARATIONS FOR THE TREATMENT OF CARBON MONOXIDE POISONING

(71) Applicant: University of Pittsburgh - Of the Commonwealth System of Higher Education, Pittsburgh, PA (US)

(72) Inventors: Jason J. Rose, Fox Chapel, PA (US); Anthony W. DeMartino, Pittsburgh, PA (US); Jesus Tejero Bravo, Pittsburgh, PA (US); Mark Thomas Gladwin, Baltimore, MD (US); Matthew R. Dent, Pittsburgh, PA (US)

(73) Assignee: University of Pittsburgh - Of the Commonwealth System of Higher Education, Pittsburgh, PA (US)

(21) Appl. No.: 17/998,420

(22) PCT Filed: May 11, 2021
(86) PCT No.: PCT/US2021/031698

§ 371 Date: Nov. 10, 2022

Related U.S. Application Data
(60) Provisional application No. 63/022,821, filed on May 11, 2020.

(51) Int. Cl. C07K 14/805; A61K 9/00; A61P 39/02; A61K 45/06; A61K 35/14

(52) U.S. Cl. CPC C07K 14/805; A61K 9/0019; A61P 39/02; A61K 45/06; A61K 35/14

(57) ABSTRACT

Methods for the rapid elimination of carbon monoxide (CO) from CO-bound hemoglobin, myoglobin and cytochrome c oxidase in subjects with CO poisoning are described. The disclosed therapy involves the use of rationally designed, modified, regulator of CO metabolism (RcoM) proteins and pharmaceutical compositions thereof, which scavenge carbon monoxide from poisoned tissue. The recombinant RcoM compositions are infused into blood, where they rapidly sequester carbon monoxide and limit the toxic effects of carbon monoxide on cellular respiration, oxygen transport and oxygen utilization.

### CROSS REFERENCE TO RELATED APPLICATIONS

This application claims the benefit of U.S. Provisional Application No. 63/022,821, filed May 11, 2020, which is herein incorporated by reference in its entirety.

### ACKNOWLEDGMENT OF GOVERNMENT SUPPORT

This invention was made with government support under grant numbers HL098032, HL125886, HL136857, HL103455, HL110849 and HL007563 awarded by the National Institutes of Health. The government has certain rights in the invention.

### FIELD

This disclosure concerns recombinant regulator of carbon monoxide metabolism (RcoM) proteins and pharmaceutical compositions thereof. This disclosure further concerns use of the recombinant RcoM proteins and compositions for the treatment of carbon monoxide (CO) poisoning, cyanide poisoning and hydrogen sulfide poisoning, and as a blood substitute.

### BACKGROUND

Inhalation exposure to carbon monoxide represents a major cause of environmental poisoning. Individuals can be exposed to carbon monoxide in the air under a variety of different circumstances, such as house fires, generators or outdoor barbeque grills used indoors, or during suicide attempts in closed spaces. Carbon monoxide binds to hemoglobin and to hemoproteins in cells, in particular the enzymes of the respiratory transport chain. The accumulation of carbon monoxide bound to hemoglobin and other hemoproteins impairs oxygen delivery and oxygen utilization for oxidative phosphorylation. This ultimately results in severe hypoxic and ischemic injury to vital organs such as the brain and the heart. Individuals who accumulate greater than 5-10% carbon carboxyhemoglobin in their blood are at risk for brain injury and neurocognitive dysfunction. Patients with very high carboxyhemoglobin levels typically suffer from irreversible brain injury, respiratory failure and/or cardiovascular collapse.

Despite the availability of methods to rapidly diagnose carbon monoxide poisoning with standard arterial and venous blood gas analysis and co-oximetry, and despite an awareness of the risk factors for carbon monoxide poisoning, there are no available antidotes for this toxic exposure. The current therapy is to give 100% oxygen by face mask, and when possible, to expose patients to hyperbaric oxygen. Hyperbaric oxygen therapy increases the rate of release of carbon monoxide from hemoglobin and accelerates the natural clearance of carbon monoxide. However, this therapy has only a modest effect on carbon monoxide clearance rates and based on the complexity of hyperbaric oxygen facilities, this therapy is not available in the field. Furthermore, hyperbaric oxygen therapy is often associated with significant treatment delays and transportation costs. Thus, a need exists for an effective, rapid and readily available therapy to treat carbon monoxide poisoning, also known as carboxyhemoglobinemia.

### SUMMARY

The present disclosure describes recombinant regulator of carbon monoxide metabolism (RcoM) proteins with high affinity for CO and their use as CO scavengers. The disclosed RcoM proteins are capable of removing CO from CO-bound hemoglobin, myoglobin and cytochrome c oxidase (in mitochondria) and thus can be used in methods of treating carboxyhemoglobinemia and as blood substitutes.

Provided herein are recombinant RcoM proteins. In some embodiments, the recombinant RcoM protein includes a heme-binding domain (HBD) having an amino acid sequence that is at least 90% identical to SEQ ID NO: 2. In some examples, the amino acid sequence of the HBD is at least 90% identical to SEQ ID NO: 2 and includes an amino acid substitution at one or more of H74, C94, M104, M105, C127 and C130. In other examples, the HBD has a wild-type amino acid sequence. The recombinant RcoM protein can be a full-length RcoM (such as the RcoM of SEQ ID NO: 1), or can be a truncated RcoM, such as an RcoM consisting of or consisting essentially of a HBD. In particular examples, the recombinant RcoM protein includes an affinity tag at the N-terminus or C-terminus, such as a cleavable affinity tag.

Pharmaceutical compositions that include a recombinant RcoM protein disclosed herein are further provided. In some embodiments, the pharmaceutical composition further includes a reducing agent or an oxidizing agent.

Also provided herein is an in vitro method of removing carbon monoxide from hemoglobin, myoglobin or mitochondria (cytochrome c oxidase) in blood or animal tissue. In some embodiments, the method includes contacting the blood or animal tissue with an effective amount of a recombinant RcoM protein disclosed herein.

Further provided are methods of treating carboxyhemoglobinemia in a subject. In some embodiments, the method includes administering to the subject a therapeutically effective amount of a recombinant RcoM protein or pharmaceutical composition disclosed herein. In some examples, the recombinant RcoM protein is administered as a pharmaceutical composition comprising a reducing agent.

Also provided are methods of treating cyanide poisoning in a subject. In some embodiments, the method includes administering to the subject a therapeutically effective amount of a recombinant RcoM protein or pharmaceutical composition disclosed herein. In some examples, the RcoM protein is in its oxidized form.

Methods of treating hydrogen sulfide (H₂S) poisoning in a subject are further provided. In some embodiments, the method includes administering to the subject a therapeutically effective amount of a recombinant RcoM protein or pharmaceutical composition disclosed herein. In some examples, the RcoM protein is in its reduced form.

Further provided are methods of replacing blood in a subject. In some embodiments, the method includes administering to the subject a therapeutically effective amount of a recombinant RcoM protein or pharmaceutical composition disclosed herein.

The foregoing and other objects and features of the disclosure will become more apparent from the following detailed description, which proceeds with reference to the accompanying figures.

### BRIEF DESCRIPTION OF THE DRAWINGS

FIG. 1: The amino acid sequence of the RcoM-1 ortholog from P. xenovorans (SEQ ID NO: 1) is shown. RcoM-1 contains a PAS domain (residues 1-154 of SEQ ID NO: 1) and a LytTR domain (residues 155-267 of SEQ ID NO: 1). Also shown are the crystal structures of homologous PAS and LytTR domains from other bacteria. The PAS domain structure is from the direct oxygen sensor (DOS) protein in E. coli (Kurokawa et al., J Biol Chem 279(19): 20186-20193, 2004), and the LytTR domain structure is from the transcription factor AgrA in S. aureus (Sidote et al., Structure 16(5):727-735, 2008).

FIG. 2: The amino acid sequence of RcoM-1 from P. xenovorans truncated to include only the PAS CO-binding domain, and lacking the N-terminal methionine (residues 2-154 of SEQ ID NO: 2). Heme-binding residues are indicated in bold. Also shown is a schematic outlining the heme coordination environment in RcoM-1.

FIG. 3: The amino acid sequence of RcoM-1 from P. xenovorans further truncated to include only key regions of the PAS CO binding domain (SEQ ID NO: 3). Heme-binding residues are in bold (H74, C75 and M104, numbered with reference to SEQ ID NO: 1). Also shown is a structural alignment of the PAS domain from the E. coli DOS protein and a homology model of the RcoM-1 PAS domain, developed using the I-TASSER online modeling server. Dashes indicate the location of the proposed truncation sites. Heme-binding residues are shown as sticks.

FIG. 4: Hemoglobin-CO transfer kinetics in the presence of WT full-length RcoM-1 under aerobic conditions at 37° C., measured using stopped-flow UV-Vis spectroscopy. Concentrations of hemoglobin-CO (Hb-CO) and Fe(II) RcoM-1 were 20 μM, and experiments were performed in triplicate. The data for loss of Hb-CO was fit to a double exponential curve, which exhibited a slow-phase half-life (t_{1/2}) of 1.4 seconds. The data for increase of Fe(II)-CO RcoM was fit to a single exponential curve, which exhibited a half-life of 0.93 seconds.

FIG. 5: Hemoglobin-CO transfer kinetics in the presence of WT full-length RcoM-1 under anaerobic conditions at 37° C., measured using UV-Vis spectroscopy. Concentrations of hemoglobin-CO and Fe(II) RcoM-1 were 15 μM and 15.8 μM, respectively. Changes in absorbance at 530, 562, and 583 nm, which track transition from Fe(II) to Fe(II)-CO RcoM, were fit to a single exponential curve, which exhibited a half-life of 50 seconds.

FIG. 6: Amino acid alignment of P. xenovorans RcoM-1 (SEQ ID NO: 1) and the H. crassostreae RcoM homolog (SEQ ID NO: 4). Residues H74, C94 and M104 of P. xenovorans RcoM-1 correspond to residues H57, C75 and M85 from the H. crassostreae RcoM homolog.

FIG. 7: Comparison of UV-Vis spectra of WT RcoM-1 and HBD16 RcoM-1 containing a C94S substitution. Visible spectra for the full-length wild-type RcoM-1 (left). Visible spectra for the isolated heme binding domain (HBD) of RcoM-1 carrying the C94S mutation (right). The spectra for the ferric (Fe(III)); ferrous deoxy (Fe(II)); and ferrous-CO species (Fe(II)-CO) are shown. The tables indicate the wavelength for the peak maxima for each species (in nm) along with the estimated molar absorptivity for each peak (mM⁻¹cm⁻¹).

FIG. 8: Evidence for a stable O₂ adduct in HBD C94S. The isolated heme binding domain (HBD) of RcoM1 carrying the C94S mutation can bind to oxygen. The visible spectra for the ferrous (Fe(II)) species in the presence of the reductant sodium dithionite is indicated by *. When the reductant is removed, the Fe(II), after desalt spectrum is obtained. After air exposure, the formation of a ferrous-oxy spectrum, with maxima around 540 nm and 575 nm is observed (Fe(II), air exposure). Reoxidation of the protein yields the ferric spectrum (Fe(III), re-ox), consistent with the ferric spectrum shown in FIG. 7.

FIG. 9: Truncated HBD16 RcoM with a C94S substitution has the same CO on rate as WT RcoM. Kinetics of the reaction of the ferrous heme binding domain (HBD) of RcoM1 with carbon monoxide (CO) was determined by stopped-flow techniques. (Top left) Detail of the Soret band of the protein; arrows indicate the direction of the absorbance changes. (Top right) Detail of the visible range of the spectrum. Arrows indicate the direction of the absorbance changes. (Bottom left) Absorbance changes versus time at selected wavelengths. The calculation of the rates at different CO concentrations yields an association rate for the reaction of 1.2×10⁵ M⁻¹s⁻¹. Similar values are obtained for the wild-type, full length protein.

FIG. 10: Determination of the CO dissociation rates for the heme binding domain (HBD) of RcoM1 carrying the C94S mutation. The reaction was monitored by the absorbance change as the ferrous-CO complex dissociates in the presence of nitric oxide (NO). As CO dissociates, NO binds to the heme causing a change in the absorbance spectrum. Excess NO prevents CO from rebinding the heme. (Top left) Detail of the visible range of the spectrum. Arrows indicate the direction of the absorbance changes. (Top right) The time course of the absorbance changes allows for determination of a dissociation rate of 4.9×10⁻² s⁻¹

FIG. 11: Thermal unfolding of Fe(III) HBD RcoM-1 carrying the C94S mutation. Unfolding is monitored by the change in absorbance at the heme Soret maximum at 420 nm. The sample was allowed to equilibrate at each temperature for five minutes before recording each UV-Vis spectrum. A small loss in Soret intensity, observed between 20° C. and 75° C., was likely due to a change in heme coordination number. Loss of Soret intensity between 75° C. and 98° C. was attributed to loss of heme from the protein due to thermal unfolding. (Top left) UV-Vis spectra for Fe(III) HBD RcoM-1 bearing the C94S mutation, recorded at each temperature between 20° C. and 98° C. (Top right) Absorbance value at Soret maximum, 420 nm, as a function of temperature. (Bottom left) UV-Vis spectra recorded during thermal unfolding between 75° C. and 98° C. (Bottom right) Absorbance value at Soret maximum as a function of temperature recorded during thermal unfolding between 75° C. and 98° C. These data were used to determine a melting temperature, Tₘ, of 91° C.

FIGS. 12A-12D: Comparison of electronic absorption (UV-Vis) spectra for RcoM heme-binding domain (HBD) truncate species in WT (FIG. 12A) and Cys-replacement protein variants CC HBD (FIG. 12B), C94S (FIG. 12C) and CCC HBD (FIG. 12D). The spectra for the ferric (Fe(III)); ferrous deoxy (Fe(II)); and ferrous-CO species (Fe(II)-CO), and ferrous-oxy (Fe(II)-O₂) are displayed.

FIGS. 13A-13C: Comparison of electronic absorption (UV-Vis) spectra for RcoM HBD truncate species in Met104 variants CC M104A (FIG. 13A) and CC M104H (FIG. 13B), each bearing Cys94. The spectra for the ferric (Fe(III)); ferrous deoxy (Fe(II)); and ferrous-CO species (Fe(II)-CO), and ferrous-oxy (Fe(II)-O₂) are displayed. (FIG. 13C) Schematic for the protein-derived ligand switching mechanism for RcoM that highlights coordination sphere changes in these variants.

FIGS. 14A-14D: Comparison of electronic absorption (UV-Vis) spectra for RcoM HBD truncate species in Met104 variants CCC M104A (FIG. 14A), CCC M104L (FIG. 14B) and CCC M104H (FIG. 14C), each with the Cys94→Ser substitution. The spectra for the ferric (Fe(III)); ferrous deoxy (Fe(II)); and ferrous-CO species (Fe(II)-CO), and ferrous-oxy (Fe(II)-O₂) are displayed. (FIG. 14D) Schematic for the protein-derived ligand switching mechanism for RcoM that highlights coordination sphere changes in these variants.

FIGS. 15A-15D: Quantification of oxygen binding affinity (P₅₀) in RcoM HBD truncates. The fraction of hemoprotein bound to oxygen was measured as a function of oxygen partial pressure using UV-Vis spectroscopy using a tonometer apparatus equipped with an optical cuvette. (FIG. 15A) Representative spectral changes in UV-Vis features for CC HBD RcoM variant as a function of oxygen partial pressure, (P_{O2}). Oxygen binding curves for CC HBD (FIG. 15B), C94S HBD (FIG. 15C) and CCC HBD (FIG. 15D), plotted in terms of the fraction of oxygen-free (deoxy) hemoprotein and oxygen-bound (oxy+Fe(III)) hemoprotein. Autooxidation at low oxygen tensions likely accounts for formation of some ferric heme. Curves were fit to a non-linear, single-site binding model to quantify P₅₀.

FIGS. 16A-16D: Determination of second order rate constants for CO binding (k_{on,CO}) to RcoM WT HBD (FIG. 16A) and HBD truncates CC HBD (FIG. 16B), C94S (FIG. 16C) and CCC HBD (FIG. 16D). The CO binding rate at each concentration of CO was measured using stopped-flow UV-Vis spectroscopy and fit to a single exponential. Each data point represents an average of 2-3 replicate measurements for these rates. A linear regression was applied to each curve, and the second order rate constant was estimated as the slope.

FIGS. 17A-17C: Representative determination of autooxidation rate (k_{oxid}) for WT HBD RcoM truncate. (FIG. 17A) Reference spectra for Fe(III) and Fe(II)-O₂ proteins. (FIG. 17B) Spectral changes in UV-Vis features for Fe(II)-O₂ WT HBD. (FIG. 17C) Spectral changes at 542 nm and 573 nm were fit to a single exponential to determine k_{oxid}.

FIG. 18: Summary of ligand binding parameters and heme stability properties for WT RcoM and RcoM HBD variants C94S, CC HBD and CCC HBD.

FIGS. 19A-19B: Representative unfolding of Fe(III) CCC HBD RcoM in the presence of urea at 37° C. (FIG. 19A) Unfolding was monitored by changes in absorbance at the heme Soret maximum at 415 nm. Samples were allowed to equilibrate for 10 minutes before recording each UV-Vis spectrum. (FIG. 19B) Unfolding data were fit to a sigmoidal curve to determine the concentration of denaturant at which half of the protein sample was unfolded ([D]₅₀).

FIGS. 20A-20D: Lack of reactivity between RcoM HBD truncates and hydrogen peroxide. Fe(III) WT HBD (FIG. 20A) and variants CCC HBD (FIG. 20B), CCC M104A HBD (FIG. 20C) and CCC M104H HBD (FIG. 20D) were incubated with 500 μM hydrogen peroxide at pH 7.4, 25° C. and monitored by UV-Vis spectroscopy every 2 minutes over the course of 30 minutes. Minimal spectral changes were observed for each variant, suggesting that hydrogen peroxide does not react with the Fe(III) heme center of RcoM HBD truncates to produce highly oxidizing species.

FIGS. 21A-21C: Summary of nitrite reduction data for full-length and HBD truncate RcoM variants. Ferrous protein (10-15 μM) was incubated with 1-5 mM sodium nitrite at 37° C. in the presence of 2.5 mM sodium dithionite. (FIG. 21A) UV-Vis spectroscopy was used to monitor the conversion of Fe(II) heme to Fe(II)-NO. (FIGS. 21B-21C) Changes in spectral features at 562 nm and 578 nm were fit to a single exponential curve to determine observed rates of nitrite reduction. Observed rates were plotted as a function of nitrite concentration, a linear regression was applied to each plot with the second order rate constant estimated as the slope.

FIGS. 22A-22D: Representative kinetic traces for in vitro CO transfer from hemoglobin (Hb) to WT RcoM HBD (FIG. 22A) and RcoM HBD variants CC HBD (FIG. 22B), C94S HBD (FIG. 22C) and CCC HBD (FIG. 22D) under aerobic conditions at 37° C. CO-bound Hb (20 μM) was incubated with equimolar oxyferrous RcoM, and CO transfer from Hb to RcoM was monitored using UV-Vis spectroscopy. The fraction of each CO-bound hemoprotein was determined using spectral deconvolution, and corresponding kinetic traces were fit to a single or double exponential equation. The half-life of each CO-bound species is displayed, with the fast species half-life and amplitude displayed for curves fit to double exponentials.

FIGS. 23A-23B: Kinetic traces monitoring CO transfer from red blood cell (RBC)-encapsulated HbCO to extracellular RcoM HBD truncates under aerobic conditions at 37° C. Hemoproteins were incubated at equimolar concentrations (50-100 μM), and RBCs were separated from extracellular RcoM by centrifugation at each time point. CO transfer from Hb to WT HBD RcoM (FIG. 23A) and C94S HBD RcoM (FIG. 23B) was monitored using UV-Vis spectroscopy. The fraction of each CO-bound hemoprotein was determined using spectral deconvolution, and corresponding kinetic traces were fit to a single exponential equation. Data points represent the average of 3 trials±SEM, and the half-life of COHb in each experiment is displayed.

FIG. 24: C94S and CCC HBD RcoM variants scavenge CO from HbCO in a lethal CO poisoning model in vivo. Schematic for the in vivo model of severe CO poisoning in mice (top). Anesthetized, mechanically ventilated mice were exposed to 3,000 ppm CO in air for 4.5 minutes, followed by intravenous infusion of Fe(II)-O₂ CCC HBD RcoM at an injection volume of 10 μL/g body weight (hemoprotein concentrations listed in the table, bottom). Blood samples (15 μL) were drawn immediately before and after infusion, as well as 25 minutes after CO exposure. At each time point, RBCs were separated from plasma by centrifugation, and separated RBC pellets and plasma samples are immediately frozen at −80° C. Subsequently, the fraction of CO-bound hemoglobin from RBCs (% HbCO) and the fraction of CO-bound RcoM (% RcoM-CO) were determined using spectral deconvolution. Infusion with RcoM resulted in a greater decrease in the fraction of CO-bound Hb (Δ% HbCO) compared to infusion with PBS.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on May 3, 2021, 18.7 KB, which is incorporated by reference herein. In the accompanying sequence listing:

SEQ ID NO: 1 is the amino acid sequence of full-length WT RcoM-1 from P. xenovorans.

SEQ ID NO: 2 is the amino acid sequence of a truncated RcoM-1 lacking the LytTR domain (HBD16).

SEQ ID NO: 3 is the amino acid sequence of a truncated RcoM-1 lacking the LytTR domain and portions of the PAS domain (HBD12).

SEQ ID NO: 4 is the amino acid sequence of WT RcoM from H. crassostreae.

SEQ ID NO: 5 is the amino acid sequence of a cleavage site from tobacco etch virus (TEV).

SEQ ID NO: 6 is the amino acid sequence of a cleavage site from thrombin.

SEQ ID NO: 7 is the amino acid sequence of RcoM variant C94S HBD.

SEQ ID NO: 8 is the amino acid sequence of RcoM variant C127S/C130S HBD.

SEQ ID NO: 9 is the amino acid sequence of RcoM variant CCC HBD.

SEQ ID NO: 10 is the amino acid sequence of RcoM variant CC M104A HBD.

SEQ ID NO: 11 is the amino acid sequence of RcoM variant CC M104H HBD.

SEQ ID NO: 12 is the amino acid sequence of RcoM variant CCC M104A HBD

SEQ ID NO: 13 is the amino acid sequence of RcoM variant CCC M104H HBD.

SEQ ID NO: 14 is the amino acid sequence of RcoM variant CCC M104L HBD.

### DETAILED DESCRIPTION

### I. Abbreviations

CO carbon monoxide

H₂S hydrogen sulfide

Hb hemoglobin

Hb-CO carboxyhemoglobin

HBOC hemoglobin-based oxygen carrier

HBD heme-binding domain

NO nitric oxide

RcoM regulator of carbon monoxide metabolism

TEV tobacco etch virus

WT wild-type

### II. Terms and Methods

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various embodiments, the following explanations of terms are provided:

Administration: To provide or give a subject an agent, such as a therapeutic agent (e.g. a recombinant RcoM protein), by any effective route. Exemplary routes of administration include, but are not limited to, injection or infusion (such as subcutaneous, intramuscular, intradermal, intraperitoneal, intrathecal, intravenous, intracerebroventricular, intrastriatal, intracranial and into the spinal cord), oral, intraductal, sublingual, rectal, transdermal, intranasal, vaginal and inhalation routes.

Affinity tag: A peptide sequence that is added to a recombinant protein or polypeptide to aid in purification using an affinity-based purification technique, such as affinity chromatography. Examples of affinity tags include, but are not limited to, albumin-binding protein, alkaline phosphatase, AU1 epitope, AU5 epitope, bacteriophage T7 epitope, bacteriophage V5 epitope, biotin-carboxy carrier protein, bluetongue virus tag, calmodulin binding peptide, chloramphenicol acetyl transferase, cellulose binding domain, chitin binding domain, choline binding domain, dihydrofolate reductase, E2 epitope, FLAG epitope, galactose binding protein, green fluorescent binding protein, Glu-Glu (E-E tag), glutathione S-transferase, influenza hemagglutinin, HaloTag®, histidine affinity tag, horseradish peroxidase, HSV epitope, ketosteroid isomerase, KT3 epitope, LacZ, luciferase, maltose-binding protein, Myc epitope, NusA, PDZ domain, PDZ ligand, polyarginine, polyaspartate, polycysteine, polyhistidine, polyphenylalanine, profinity eXact, protein C, S1-tag, S tag, Staphylococcal protein A (protein A), Staphylococcal protein G (protein G), Strep-tag, streptavidin, small ubiquitin-like modifier (SUMO), thioredoxin, TrpE, ubiquitin, and VSV-G (see, for example, Kimple et al., Curr Protoc Protein Sci 73: 9.9.1-.9.9.23, 2013, doi:10.1002/0471140864.ps0909s73).

Anemia: A deficiency of red blood cells and/or hemoglobin. Anemia is the most common disorder of the blood, and it results in a reduced ability of blood to transfer oxygen to the tissues. Since all human cells depend on oxygen for survival, varying degrees of anemia can have a wide range of clinical consequences. The three main classes of anemia include excessive blood loss (acutely such as a hemorrhage or chronically through low-volume loss), excessive blood cell destruction (hemolysis) or deficient red blood cell production (ineffective hematopoiesis).

The term “anemia” refers to all types of clinical anemia, including but not limited to: microcytic anemia, iron deficiency anemia, hemoglobinopathies, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle-cell anemia, warm autoimmune hemolytic anemia, cold agglutinin hemolytic anemia.

In severe cases of anemia, or with ongoing blood loss, a blood transfusion may be necessary. Doctors may use any of a number of clinically accepted criteria to determine that a blood transfusion is necessary to treat a subject with anemia. For instance, the currently accepted Rivers protocol for early goal-directed therapy for sepsis requires keeping the hematocrit above 30.

Anoxia: A pathological condition in which the body as a whole or region of the body is completely deprived of oxygen supply.

Antidote: An agent that neutralizes or counteracts the effects of a poison, such as carbon monoxide.

Bleeding disorder: A general term for a wide range of medical problems that lead to poor blood clotting and continuous bleeding. Physicians also refer to bleeding disorders by terms such as, for example, coagulopathy, abnormal bleeding and clotting disorders. Bleeding disorders include any congenital, acquired or induced defect that results in abnormal (or pathological) bleeding. Examples include, but are not limited to, disorders of insufficient clotting or hemostasis, such as hemophilia A (a deficiency in Factor VIII), hemophilia B (a deficiency in Factor IX), hemophilia C (a deficiency in Factor XI), other clotting factor deficiencies (such as Factor VII or Factor XIII), abnormal levels of clotting factor inhibitors, platelet disorders, thrombocytopenia, vitamin K deficiency and von Willebrand's disease.

Bleeding episode: Refers to an occurrence of uncontrolled, excessive and/or pathological bleeding. Bleeding episodes can result from, for example, drug-induced bleeding (such as bleeding induced by non-steroidal anti-inflammatory drugs or warfarin), anticoagulant overdose or poisoning, aneurysm, blood vessel rupture, surgery and traumatic injury (including, for example, abrasions, contusions, lacerations, incisions or gunshot wounds). Bleeding episodes can also result from diseases such as cancer, gastrointestinal ulceration or from infection.

Blood replacement product or blood substitute: A composition used to fill fluid volume and/or carry oxygen and other blood gases in the cardiovascular system. Blood substitutes include, for example, volume expanders (to increase blood volume) and oxygen therapeutics (to transport oxygen in blood). Oxygen therapeutics include, for example, hemoglobin-based oxygen carriers (HBOC) and perfluorocarbons (PFCs). A preferred blood substitute is one that mimics the oxygen-carrying capacity of hemoglobin, requires no cross-matching or compatibility testing, with a long shelf life, exhibits a long intravascular half-life (over days and weeks), and is free of side effects and pathogens.

Carbon monoxide (CO): A colorless, odorless and tasteless gas that is toxic to humans and animals when encountered at sufficiently high concentrations. CO is also produced during normal animal metabolism at low levels.

Carboxyhemoglobin (HbCO): A stable complex of carbon monoxide (CO) and hemoglobin (Hb) that forms in red blood cells when CO is inhaled or produced during normal metabolism.

Carboxyhemoglobinemia or carbon monoxide poisoning: A condition resulting from the presence of excessive amounts of carbon monoxide in the blood. Typically, exposure to CO of 100 parts per million (ppm) or greater is sufficient to cause carboxyhemoglobinemia. Symptoms of mild acute CO poisoning include lightheadedness, confusion, headaches, vertigo, and flu-like effects; larger exposures can lead to significant toxicity of the central nervous system and heart, and even death. Following acute poisoning, long-term sequelae often occur. Carbon monoxide can also have severe effects on the fetus of a pregnant woman. Chronic exposure to low levels of carbon monoxide can lead to depression, confusion, and memory loss. Carbon monoxide mainly causes adverse effects in humans by combining with hemoglobin to form carboxyhemoglobin (HbCO) in the blood. This prevents oxygen binding to hemoglobin, reducing the oxygen-carrying capacity of the blood, leading to hypoxia. Additionally, myoglobin and mitochondrial cytochrome oxidase are thought to be adversely affected. Carboxyhemoglobin can revert to hemoglobin, but the recovery takes time because the HbCO complex is fairly stable. Current methods of treatment for CO poisoning including administering 100% oxygen or providing hyperbaric oxygen therapy.

Cerebral ischemia or ischemic stroke: A condition that occurs when an artery to or in the brain is partially or completely blocked such that the oxygen demand of the tissue exceeds the oxygen supplied. Deprived of oxygen and other nutrients following an ischemic stroke, the brain suffers damage as a result of the stroke.

Coagulopathy: A medical term for a defect in the body's mechanism for blood clotting.

Contacting: Placement in direct physical association; includes both in solid and liquid form. When used in the context of an in vivo method, “contacting” also includes administering.

Cyanide poisoning: A type of poisoning that results from exposure to some forms of cyanide, such as hydrogen cyanide gas and cyanide salt. Cyanide poisoning can occur from inhaling smoke from a house fire, exposure to metal polishing, particular insecticides and certain seeds (such as apple seeds). Early symptoms of cyanide poisoning include headache, dizziness, rapid heart rate, shortness of breath and vomiting. Later symptoms include seizures, slow heart rate, low blood pressure, loss of consciousness and cardiac arrest.

Cytochrome c oxidase: An enzyme that is part of the respiratory electron transport chain. This enzyme is found in the mitochondria.

Favism: The common name of glucose-6-phosphate dehydrogenase (G6PD) deficiency; an X-linked recessive hereditary disease featuring non-immune hemolytic anemia in response to a number of causes.

Fusion protein: A protein comprising at least a portion of two different (heterologous) proteins.

Gastrointestinal bleeding: Refers to any form of hemorrhage (loss of blood) in the gastrointestinal tract, from the pharynx to the rectum.

Hemoglobin (Hb): The iron-containing oxygen-transport metalloprotein in the red blood cells of the blood in vertebrates and other animals. In humans, the hemoglobin molecule is an assembly of four globular protein subunits. Each subunit is composed of a protein chain tightly associated with a non-protein heme group. Each protein chain arranges into a set of alpha-helix structural segments connected together in a globin fold arrangement, so called because this arrangement is the same folding motif used in other heme/globin proteins. This folding pattern contains a pocket which strongly binds the heme group.

Hemoglobin-based oxygen carrier (HBOC): A transfusable fluid of purified, recombinant and/or modified hemoglobin that functions as an oxygen carrier and can be used as a blood substitute. A number of HBOCs are known and/or in clinical development. Examples of HBOCs include, but are not limited to, DCLHb (HEMASSIST™; Baxter), MP4 (HEMOSPAN™; Sangart), pyridoxylated Hb POE−conjugate (PHP)+catalase & SOD (Apex Biosciences), O—R—PolyHbA₀ (HEMOLINK™; Hemosol), PolyBvHb (HEMOPURE™; Biopure), PolyHb (POLYHEME™; Northfield), rHb1.1 (OPTRO™; Somatogen), PEG-Hemoglobin (Enzon), OXYVITA™ and HBOC-201 (Greenburg and Kim, Crit Care 8(Suppl 2):561-S64, 2004; to Lintel Hekkert et al., Am J Physiol Heart Circ Physiol 298:H1103-H1113, 2010; Eisenach, Anesthesiology 111:946-963, 2009).

Hemophilia: The name of several hereditary genetic illnesses that impair the body's ability to control coagulation.

Hemorrhage: The loss of blood from the circulatory system. Bleeding can occur internally, where blood leaks from blood vessels inside the body, or externally, either through a natural opening such as the vagina, mouth or rectum, or through a break in the skin.

Heterologous: A heterologous protein or polypeptide refers to a protein or polypeptide derived from a different source or species.

Hydrogen sulfide poisoning: A type of poisoning resulting from excess exposure to hydrogen sulfide (H₂S). H₂S binds iron in the mitochondrial cytochrome enzymes and prevents cellular respiration. Exposure to lower concentrations of H₂S can cause eye irritation, sore throat, coughing, nausea, shortness of breath, pulmonary edema, fatigue, loss of appetite, headaches, irritability, poor memory and dizziness. Higher levels of exposure can cause immediate collapse, inability to breath and death.

Hemorrhagic shock: A condition of reduced tissue perfusion, resulting in the inadequate delivery of oxygen and nutrients that are necessary for cellular function. Hypovolemic shock, the most common type, results from a loss of circulating blood volume from clinical etiologies, such as penetrating and blunt trauma, gastrointestinal bleeding, and obstetrical bleeding.

Hypoxaemia: An abnormal deficiency in the concentration of oxygen in arterial blood.

Hypoxia: A pathological condition in which the body as a whole (generalized hypoxia) or region of the body (tissue hypoxia) is deprived of adequate oxygen supply.

Ischemia: A vascular phenomenon in which a decrease in the blood supply to a bodily organ, tissue, or part is caused, for instance, by constriction or obstruction of one or more blood vessels. Ischemia sometimes results from vasoconstriction or thrombosis or embolism. Ischemia can lead to direct ischemic injury, tissue damage due to cell death caused by reduced oxygen supply.

Ischemia/reperfusion injury: In addition to the immediate injury that occurs during deprivation of blood flow, ischemic/reperfusion injury involves tissue injury that occurs after blood flow is restored. Current understanding is that much of this injury is caused by chemical products and free radicals released into the ischemic tissues.

When a tissue is subjected to ischemia, a sequence of chemical events is initiated that may ultimately lead to cellular dysfunction and necrosis. If ischemia is ended by the restoration of blood flow, a second series of injurious events ensue, producing additional injury. Thus, whenever there is a transient decrease or interruption of blood flow in a subject, the resultant injury involves two components—the direct injury occurring during the ischemic interval and the indirect or reperfusion injury that follows. When there is a long duration of ischemia, the direct ischemic damage, resulting from hypoxia, is predominant. For relatively short duration ischemia, the indirect or reperfusion mediated damage becomes increasingly important. In some instances, the injury produced by reperfusion can be more severe than the injury induced by ischemia per se. This pattern of relative contribution of injury from direct and indirect mechanisms has been shown to occur in all organs.

Isolated: An “isolated” biological component (such as a nucleic acid molecule, protein, or cell) has been substantially separated or purified away from other biological components in the cell, blood or tissue of the organism, or the organism itself, in which the component naturally occurs, such as other chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins.

Methemoglobin: The oxidized form of hemoglobin in which the iron in the heme component has been oxidized from the ferrous (+2) to the ferric (+3) state. This renders the hemoglobin molecule incapable of effectively transporting and releasing oxygen to the tissues. Normally, there is about 1% of total hemoglobin in the methemoglobin form.

Microcytosis: A blood disorder characterized by the presence of microcytes (abnormally small red blood cells) in the blood.

Myoglobin: A heme-containing globin protein found in the muscle tissue of vertebrates and most mammals. Myoglobin carries and stores oxygen in muscle cells.

Oxidizing agent: A substance that is capable of accepting an electron from another substance (also referred to as “oxidizing” a substance). An oxidizing agent gains electrons and is reduced in a chemical reaction. An oxidizing agent is also known as an “electron acceptor.” In some embodiments herein, the oxidizing agent is a quinone, such as benzoquinone or napthaquinone. In other embodiments, the oxidizing agent is an oxygen-containing gas mixture, an oxygen-containing liquid mixture, a ferricyanide salt, or any combination thereof. In some examples, an electron mediator (e.g. TMPD or crystal violet) is used in combination with an oxidizing agent in order to facilitate electron transfer. In some embodiments herein, oxidation of RcoM is carried out by exposure to visible light.

Paraburkholderia xenovorans: A species of proteobacteria found in the soil. P. xenovorans is a Gram-negative aerobic bacterium. P. xenovorans has one of the largest known prokaryotic genomes at 9.7 Mb. This bacteria is capable of efficiently degrading polychlorinated biphenyl (PCB). P. xenovorans is also known as Burkholderia xenovorans.

Peptide or Polypeptide: A polymer in which the monomers are amino acid residues which are joined together through amide bonds. When the amino acids are alpha-amino acids, either the L-optical isomer or the D-optical isomer can be used, the L-isomers being preferred. The terms “peptide,” “polypeptide” or “protein” as used herein are intended to encompass any amino acid sequence and include modified sequences, including modified RcoM proteins. The terms “peptide” and “polypeptide” are specifically intended to cover naturally occurring proteins, as well as those which are recombinantly or synthetically produced.

Conservative amino acid substitutions are those substitutions that, when made, least interfere with the properties of the original protein, that is, the structure and especially the function of the protein is conserved and not significantly changed by such substitutions. Examples of conservative substitutions are shown in the following table.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamine or aspartic acid; or (d) a residue having a bulky side chain, for example, phenylalanine, is substituted for (or by) one not having a side chain, for example, glycine.

Pharmaceutically acceptable carriers: The pharmaceutically acceptable carriers of use are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the proteins and other compositions disclosed herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in HbCO in the blood of a subject with CO poisoning. “Ameliorating” refers to the reduction in the number or severity of signs or symptoms of a disease.

Purified: The term purified does not require absolute purity; rather, it is intended as a relative term. Thus, for example, a purified peptide preparation is one in which the peptide or protein is more enriched than the peptide or protein is in its natural environment within a cell. In one embodiment, a preparation is purified such that the protein or peptide represents at least 50% of the total peptide or protein content of the preparation. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Recombinant: A recombinant nucleic acid or protein is one that has a sequence that is not naturally occurring or has a sequence that is made by an artificial combination of two otherwise separated segments of sequence. This artificial combination is often accomplished by chemical synthesis or by the artificial manipulation of isolated segments of nucleic acids, for example, by genetic engineering techniques. The term recombinant includes nucleic acids and proteins that have been altered by addition, substitution, or deletion of a portion of a natural nucleic acid molecule or protein.

Reducing agent: An element or compound that loses (or “donates”) an electron to another chemical species in a redox chemical reaction. A reducing agent is typically in one of its lower possible oxidation states, and is known as the electron donor. A reducing agent is oxidized, because it loses electrons in the redox reaction. Exemplary reducing agents include, but are not limited to, sodium dithionite, ascorbic acid, N-acetylcysteine, methylene blue, glutathione, cytochrome b5/b5-reductase, hydralazine, earth metals, formic acid and sulfite compounds.

Regulator of carbon monoxide metabolism (RcoM): A protein found in some prokaryotes involved in CO sensing and transcriptional regulation. RcoM proteins contain an N-terminal PAS domain and a DNA-binding LytTR domain. The PAS domain contains a hexacoordinated b-type heme moiety that avidly binds CO and nitric oxide (NO). Residues His74 and Met104 of the PAS domain serve as the heme Fe(II) axial ligands, with displacement of Met104 upon binding of CO or NO. The aerobic Gram-negative bacterium Paraburkholderia xenovorans (also known as Burkholderia xenovorans) expresses two homologous proteins, RcoM-1 and RcoM-2, which share approximately 93% sequence identity and have a very high affinity for CO. RcoM-1 and RcoM-2 act as CO sensors capable of regulating aerobic CO oxidation and anaerobic CO oxidation. The wild-type amino acid sequence of P. xenovorans RcoM-1 is set forth herein as SEQ ID NO: 1. RcoM homologs (and UniProt IDs) from a variety of bacterial species are listed in Table 3.

Rhabdomyolysis: The rapid breakdown of skeletal muscle tissue due to traumatic injury, including mechanical, physical or chemical. The principal result is a large release of the creatine phosphokinase enzymes and other cell byproducts into the blood system and acute renal failure due to accumulation of muscle breakdown products, several of which are injurious to the kidney.

Sequence identity/similarity: The identity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI) and on the internet, for use in connection with the sequence analysis programs blastp, blastn, blastx, tblastn and tblastx. Additional information can be found at the NCBI web site.

Spherocytosis: An auto-hemolytic anemia characterized by the production of red blood cells (or erythrocytes) that are sphere-shaped, rather than donut-shaped.

Subject: Living multi-cellular organisms, including vertebrate organisms, a category that includes both human and non-human mammals

Thalassemia: An inherited autosomal recessive blood disease. In thalassemia, the genetic defect results in reduced rate of synthesis of one of the globin chains that make up hemoglobin. Reduced synthesis of one of the globin chains causes the formation of abnormal hemoglobin molecules, and this in turn causes the anemia which is the characteristic presenting symptom of the thalassemias.

Therapeutically effective amount: A quantity of compound or composition, for instance, an isolated or recombinant RcoM protein, sufficient to achieve a desired effect in a subject being treated. For instance, this can be the amount necessary to scavenge carbon monoxide in the blood or tissues, reduce the level of HbCO in the blood, and/or reduce one or more signs or symptoms associated with carbon monoxide poisoning.

Ulcer: An open sore of the skin, eyes or mucous membrane, often caused, but not exclusively, by an initial abrasion and generally maintained by an inflammation, an infection, and/or medical conditions which impede healing.

Vasospasm: One cause of stroke; occurs secondary to spasm of blood vessels supplying the brain. This type of stroke typically follows a subarachnoid aneurismal hemorrhage with a delayed development of vasospasm within 2-3 weeks of the bleeding event. A similar type of stroke may complicate sickle cell disease.

### IV. Recombinant RcoM Proteins

A need exists for an effective, rapid and readily available therapy to treat carboxyhemoglobinemia. The present disclosure provides recombinant regulator of carbon monoxide metabolism (RcoM) proteins that exhibit very high affinity for carbon monoxide, and thus can be used as CO scavengers. The disclosed RcoM proteins can also be used to treat hydrogen sulfide or cyanide poisoning, or can be used as blood substitutes.

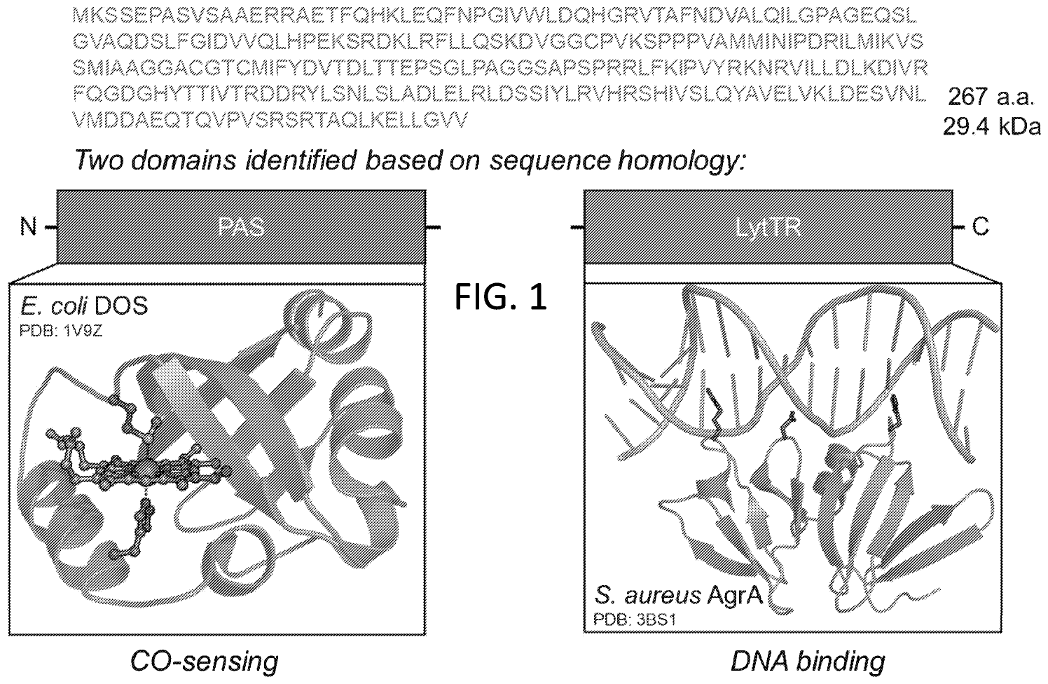

RcoM proteins were first identified as CO-sensing bacterial transcriptional regulators that couple an N-terminal PAS fold domain to a C-terminal DNA-binding LytTR domain (see FIG. 1). RcoM proteins contain a hexacoordinated b-type heme moiety that avidly binds CO and nitric oxide (NO). PAS domain residues His74 and Met104 (with respect to SEQ ID NO: 1) serve as the heme Fe(II) axial ligands, with displacement of Met104 upon binding of CO or NO. Two RcoM homologs from P. xenovorans (RcoM-1 and RcoM-2) are functional in vivo, and act as CO sensors capable of regulating aerobic CO oxidation and anaerobic CO oxidation.

RcoM exhibits very high affinity for CO and is selective for CO over oxygen. In view of these properties, the disclosed RcoM proteins are ideal for scavenging CO directly from CO-bound hemoglobin, myoglobin and cytochrome c oxidase to treat carbon monoxide poisoning. The disclosed RcoM proteins can be also used to treat cyanide or H₂S poisoning, or as blood substitutes. Further described herein are directed mutations to enhance stability, increase CO affinity, and/or lower oxygen affinity of the RcoM proteins.

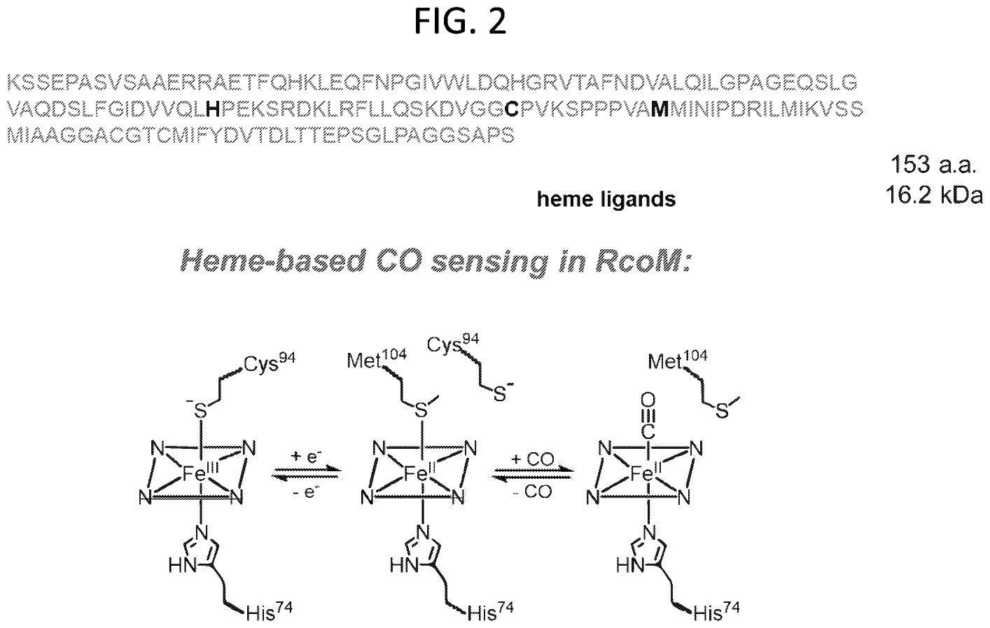

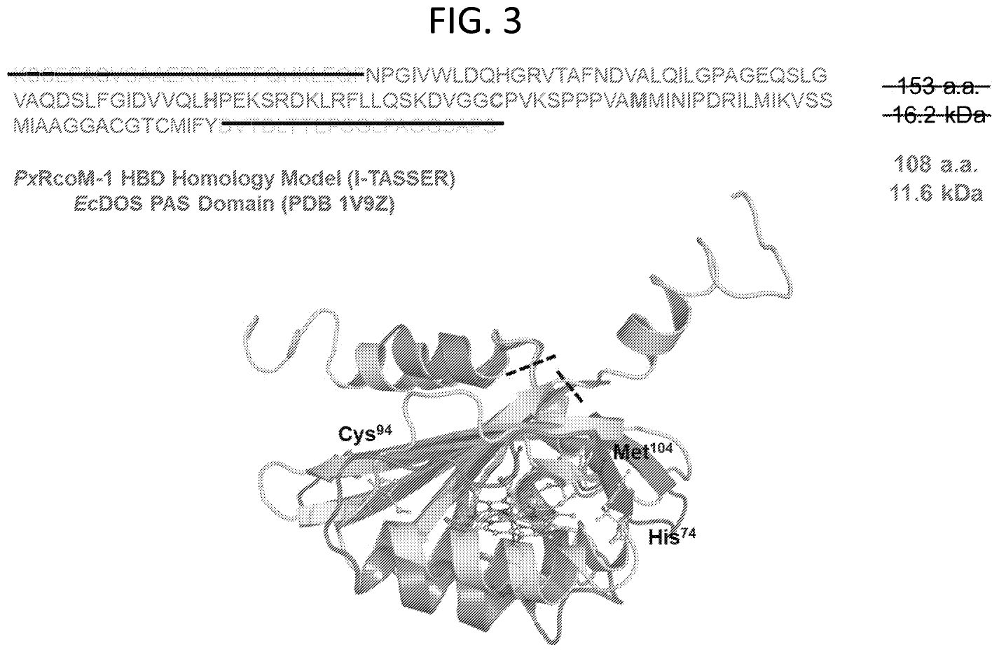

Wild-type (WT) and modified RcoM proteins are described below. In the WT amino acid sequence (SEQ ID NO: 1), the LytTR domain (DNA-binding) is underlined; the remainder of the sequence is the PAS domain (see FIG. 1). The truncated RcoM proteins disclosed herein (SEQ ID NOs: 2, 3 and 7-14) do not contain the LytTR domain (see FIGS. 2 and 3). In all RcoM sequences (SEQ ID NOs: 1-3 and 7-14), the residues in bold correspond to H74, C94, M104, C127, C130 and M105, numbered with respect to SEQ ID NO: 1.

WT RcoM-1 from P. xenovorans (29 kDa):
(SEQ ID NO: 1)
MKSSEPASVSAAERRAETFQHKLEQFNPGIVWLDQ
HGRVTAFNDVALQILGPAGEQSLGVAQDSLFGIDV
VQLHPEKSRDKLRFLLQSKDVGGCPVKSPPPVAMM
INIPDRILMIKVSSMIAAGGACGTCMIFYDVTDLT
TEPSGLPAGGSAPSPRRLFKIPVYRKNRVILLDLK
DIVRFQGDGHYTTIVTRDDRYLSNLSLADLELRLD
SSIYLRVHRSHIVSLQYAVELVKLDESVNLVMDDA
EQTQVPVSRSRTAQLKELLGVV
HBD16 RcoM (16 kDa) truncate:
(SEQ ID NO: 2)
MKSSEPASVSAAERRAETFQHKLEQFNPGIVWLDQ
HGRVTAFNDVALQILGPAGEQSLGVAQDSLFGIDV
VQLHPEKSRDKLRFLLQSKDVGGCPVKSPPPVAMM
INIPDRILMIKVSSMIAAGGACGTCMIFYDVTDLT
TEPSGLPAGGSAPS
HBD12 RcoM (12 kDa) truncate:
(SEQ ID NO: 3)
NPGIVWLDQHGRVTAFNDVALQILGPAGEQSLGVA
QDSLFGIDVVQLHPEKSRDKLRFLLQSKDVGGCPV
KSPPPVAMMINIPDRILMIKVSSMIAAGGACGTCM
IFY

Throughout this disclosure, except where indicated otherwise, specific amino acid residues are numbered with reference to full-length WT RcoM-1 of SEQ ID NO: 1. Table 1 lists the location of each corresponding residue in SEQ ID NOs: 1-3.

TABLE 1
Key residues in WT and truncated RcoM sequences

| WT RcoM | HBD16 RcoM | HBD12 RcoM |
|---|---|---|
| (SEQ ID NO: 1) | (SEQ ID NO: 2) | (SEQ ID NO: 3) |
| H73 | H73 | H48 |
| C93 | C93 | C68 |
| M103 | M103 | M78 |
| M104 | M104 | M79 |
| C126 | C126 | C101 |
| C129 | C129 | C104 |

Eight RcoM HBD variants were generated based on HBD16 of SEQ ID NO: 2. Table 2 lists each variant, along with their respective amino acid substitutions and complete amino acid sequences (residues in bold indicate substitutions).

TABLE 2
RcoM HBD16 variants

| | Amino acid | SEQ ID | |
|---|---|---|---|
| Protein name | substitutions | NO: | Sequence |
| WT HBD | none | 2 | MKSSEPASVSAAERRAETFQHKLEQFNPGIV |
| | | | WLDQHGRVTAFNDVALQILGPAGEQSLGVA |
| | | | QDSLFGIDVVQLHPEKSRDKLRFLLQSKDVG |
| | | | GCPVKSPPPVAMMINIPDRILMIKVSSMIAAG |
| | | | GACGTCMIFYDVTDLTTEPSGLPAGGSAPS |
| C94S HBD | Cys94→Ser | 7 | MKSSEPASVSAAERRAETFQHKLEQFNPGIV |
| | | | WLDQHGRVTAFNDVALQILGPAGEQSLGVA |
| | | | QDSLFGIDVVQLHPEKSRDKLRFLLQSKDVG |
| | | | GSPVKSPPPVAMMINIPDRILMIKVSSMIAAG |
| | | | GACGTCMIFYDVTDLTTEPSGLPAGGSAPS |
| C127S/C130S | Cys127→Ser | 8 | MKSSEPASVSAAERRAETFQHKLEQFNPGIV |
| HBD | Cys130→Ser | | WLDQHGRVTAFNDVALQILGPAGEQSLGVA |
| | | | QDSLFGIDVVQLHPEKSRDKLRFLLQSKDVG |
| | | | GCPVKSPPPVAMMINIPDRILMIKVSSMIAAG |
| | | | GASGTSMIFYDVTDLTTEPSGLPAGGSAPS |
| CCC HBD | Cys94→Ser | 9 | MKSSEPASVSAAERRAETFQHKLEQFNPGIV |
| | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GSPVKSPPPVAMMINIPDRILMIKVSSMIAAG |
| | | | GASGTSMIFYDVTDLTTEPSGLPAGGSAPS |
| CCM104A | Met104→Ala | 10 | MKSSEPASVSAAERRAETFQHKLEQFNPGIV |
| HBD | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GCPVKSPPPVAAMINIPDRILMIKVSSMIAAG |
| | | | GASGTSMIFYDVTDLTTEPSGLPAGGSAPS |
| CCM104H | Met104→His | 11 | MKSSEPASVSAAERRAETFQHKLEQFNPGIV |
| HBD | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GCPVKSPPPVAHMINIPDRILMIKVSSMIAAG |
| | | | GASGTSMIFYDVTDLTTEPSGLPAGGSAPS |
| CCC Ml04A | Met104→Ala | 12 | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVAAMINIPDRILMIKVSSMIAAG |
| | | | GASGTSMIFYDVTDLTTEPSGLPAGGSAPS |
| CCC M104H | Met104→His | 13 | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVAHMINIPDRILMIKVSSMIAAG |
| | | | GASGTSMIFYDVTDLTTEPSGLPAGGSAPS |
| CCC M104L | Met104→Leu | 14 | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVALMINIPDRILMIKVSSMIAAG |
| | | | GASGTSMIFYDVTDLTTEPSGLPAGGSAPS |

Provided herein are recombinant regulator of carbon monoxide metabolism (RcoM) proteins that exhibit very high affinity for CO. In some embodiments, the recombinant RcoM protein includes a heme-binding domain (HBD), and the amino acid sequence of the HBD is at least 80%, at least 85%, at least 90%, at least 95%, at least 96%, at least 97%, at least 98% or at least 99% identical to SEQ ID NO: 2. In some embodiments, the amino acid sequence of the HBD is a wild-type sequence (such as SEQ ID NO: 2). In other embodiments, the amino acid sequence of the HBD comprises an amino acid substitution at one or more of H74, C94, M104, M105, C127 and C130. In some examples, the amino acid sequence of the HBD is at least 90% or at least 95% identical to SEQ ID NO: 2 and includes an amino acid substitution at one or more of C94, M104, C127 and C130.

The disclosed RcoM proteins can be modified, such as by amino acid substitution at a variety of residues, in order to alter heme ligand affinity and/or specificity, and/or to enhance protein stability. In some embodiments, the RcoM protein includes a single amino acid substitution. In other embodiments, the RcoM protein includes at least two, at least three, at least four, at least 5 or at least 6 amino acid substitutions. In some examples, the amino acid substitutions are conservative substitutions.

In some examples, the recombinant RcoM protein includes a substitution at H74, which is a heme-coordinating histidine. In specific non-limiting examples, the substitution is selected from H74S, H74T, H74M, H74W, H74A, H74L, H74I, H74V and H74G.

In some examples, the recombinant RcoM protein includes a substitution at C94, which is a Fe(II) heme-coordinating cysteine. In specific non-limiting examples, the substitution is selected from C94S, C94T, C94H, C94W, C94M, C94A, C94L, C94I, C94V and C94G.

In some examples, the recombinant RcoM protein includes a substitution at M104, which is a Fe(II) heme-coordinating methionine. In specific non-limiting examples, the substitution is selected from M104S, M104T, M104H, M104W, M104A, M104L, M104I, M104V and M104G.

In some examples, the recombinant RcoM protein includes a substitution at M105, which is a non-heme-coordinating methionine. In specific non-limiting examples, the substitution is selected from M105S, M105T, M105H, M105W, M105A, M105L, M105I, M105V and M105G.

In some examples, the recombinant RcoM protein includes a substitution at C127, which is a non-heme-coordinating cysteine. In specific non-limiting examples, the substitution is selected from C127S, C127T, C127M, C127A, C127L, C127I, C127V and C127G.

In some examples, the recombinant RcoM protein includes a substitution at C130, which is a non-heme-coordinating cysteine. In specific non-limiting examples, the substitution is selected from C130S, C130T, C130M, C130A, C130L, C130I, C130V and C130G.

In some examples, the recombinant RcoM protein includes: a single amino acid substitution at C94; a single amino acid substitution at M104; two amino acid substitutions at C94 and M104; two amino acid substitutions at C127 and C130; three amino acid substitutions at C94, C127 and C130; three amino acid substitutions at M104, C127 and C130; three amino acid substitutions at H74, C94 and M104; four amino acid substitutions at C94, M104, C127 and C130; five amino acid substitutions at C94, M104, M105, C127 and C130; five amino acid substitutions at H74, C94, M104, C127 and C130; or six amino acid substitutions at H74, C94, M104, M105, C127 and C130. In specific non-limiting examples, the recombinant RcoM protein includes a C94S substitution; a C127S substitution and a C130S substitution; a C94S substitution, a C127S substitution and a C130S substitution; a C94S substitution and a M104L substitution; a M104A substitution, a C127S substitution and a C130S substitution; a M104H substitution, a C127S substitution and a C130S substitution; a M104L substitution, a C127S substitution and a C130S substitution; a C94S substitution, a M104A substitution, a C127S substitution and a C130S substitution; a C94S substitution, a M104H substitution, a C127S substitution and a C130S substitution; a C94S substitution, a M104L substitution, a C127S substitution and a C130S substitution; a H74S substitution, a C94S substitution and a M104L substitution; a C94S substitution, a M104L substitution, a M105L substitution, a C127S substitution and a C130S substitution; or a H74S substitution, a C94S substitution, a M104L substitution, a M105L substitution, a C127S substitution and a C130S substitution.

In particular examples, the amino acid sequence of the RcoM protein comprises or consists of SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14.

In some embodiments, the RcoM protein has an amino acid sequence at least 80%, at least 85%, at least 90%, at least 95%, at least 96%, at least 97%, at least 98% or at least 99% identical to any one of SEQ ID NOs: 1-3. In some examples, the RcoM protein comprises or consists of any one of SEQ ID NOs: 1-3.

In some examples, the amino acid sequence of the RcoM protein comprises or consists of the amino acid sequence of SEQ ID NO: 1, SEQ ID NO: 2 or SEQ ID NO: 3, except for an amino acid substitution at one or more of H74, C94, M104, C127, C130 and M105.

In specific examples, the amino acid sequence of the RcoM protein consists of SEQ ID NO: 1 except for a H74S substitution, a C94S substitution, a M104 substitution selected from M104A, M104H and M104L, a M105L substitution, a C127S substitution, a C130S substitution, or any combination thereof. In other examples, the amino acid sequence of the protein consists of SEQ ID NO: 2 except for a H74S substitution, a C94S substitution, a M104 substitution selected from M104A, M104H and M104L, a M105L substitution, a C127S substitution, a C130S substitution, or any combination thereof. In yet other specific examples, the amino acid sequence of the protein consists of SEQ ID NO: 3 except for a H74S substitution, a C94S substitution, a M104 substitution selected from M104A, M104H and M104L, a M105L substitution, a C127S substitution, a C130S substitution, or any combination thereof.

Using bioinformatics analysis, 112 rcoM genes in a variety of microorganisms were identified, 44 of which are associated with aerobic CO metabolism. One of the identified rcoM genes is from a mesophilic microorganism (Hydrogenophaga crassostreae), which is believed to express a RcoM protein with enhanced thermal stability. Thus, in some embodiments, the recombinant RcoM protein is a protein from one of the species listed in Table 3 and having the listed UniProt ID.

TABLE 3
Microorganisms having rcoM gene homologs

| Organism | UniProt ID |
|---|---|
| Comamonadaceae bacterium A1 | A0A060NIX7 |
| Cupriavidus sp. SK-3 | A0A069I1C5 |
| Burkholderiaceae bacterium 16 | A0A0F0G0P7 |
| beta proteobacterium AAP51 | A0A0N1AP15 |
| Curvibacter sp. PAE-UM | A0A0R0MH04 |
| Grimontia marina | A0A128EZU2 |
| Paraburkholderia monticola | A0A149PAY8 |
| Hydrogenophaga crassostreae | A0A162SSS9 |
| Variovorax sp. HW608 | A0A1C6R2V7 |
| Rubrivivax sp. SCN 70-15 | A0A1E4NRK7 |
| Burkholderiales bacterium GWF1_66_17 | A0A1F4H2I3 |
| Curvibacter sp. GWA2_64_110 | A0A1F8VIB7 |
| Burkholderia sp. TNe-862 | A0A1G6SJT8 |
| Paraburkholderia phenazinium | A0A1G8AQL6 |
| Burkholderia sp. yr281 | A0A1G8R819 |
| Variovorax sp. YR216 | A0A1H4GT06 |
| Paraburkholderia caballeronis | A0A1H7JEL3 |
| Aquisalimonas asiatica | A0A1H8S1B5 |
| mine drainage metagenome | A0A1J5RDU8 |
| mine drainage metagenome | A0A1J5REQ7 |
| mine drainage metagenome | A0A1J5RS58 |
| Paraburkholderia aromaticivorans | A0A248VYY9 |
| Comamonadaceae bacterium PBBC1 | A0A257EGX4 |
| Acidocella sp. 20-57-95 | A0A257Q5N9 |
| Acidiphilium sp. 21-60-14 | A0A257S620 |
| Polaromonas sp. 35-63-240 | A0A258QFN4 |
| Thiomonas sp. 15-66-11 | A0A259PBK4 |
| Burkholderia sp. IDO3 | A0A2A4CH08 |
| Massilia eurypsychrophila | A0A2G8TF36 |
| Limnohabitans sp. B9-3 | A0A2M6VJM2 |
| Limnohabitans sp. 15K | A0A2M6VYD3 |
| Betaproteobacteria bacterium HGW- | A0A2N2RI10 |
| Betaproteobacteria-3 | |
| Betaproteobacteria bacterium HGW- | A0A2N2UTK3 |
| Betaproteobacteria-11 | |
| Burkholderiales bacterium | A0A2N9LWB1 |
| Paraburkholderia eburnea | A0A2S4MA17 |
| Limnohabitans planktonicus II-D5 | A0A2T7UBD9 |
| Paraburkholderia silvatlantica | A0A2U1A6A7 |
| Spiribacter sp. E85 | A0A2U2N030 |
| Paraburkholderia sp. PDC91 | A0A2W7FSX8 |
| Burkholderia sp. H160 | B5WBI7 |
| Oxalobacteraceae bacterium IMCC9480 | F1VWB2 |
| Burkholderia sp. Ch1-1 | I2IKA5 |
| Alkalilimnicola ehrlichii (strain ATCC BAA- | Q0A8C9 |
| 1101/DSM 17681/MLHE-1) | |
| Paraburkholderia xenovorans (strain LB400) | Q13IY4 |
| Paraburkholderia xenovorans (strain LB400) | Q13YL3 |
| Betaproteobactera bacterium MOLA814 | V4YJL6 |
| Limnohabitans sp. MMS-10A-192 | A0A315BF48 |
| Limnohabitans sp. MMS-10A-160 | A0A315BYJ0 |
| Limnohabitans sp. Jir72 | A0A315E3S2 |
| Limnohabitans sp. 2KL-1 | A0A315FBI5 |
| Sinimarinibacterium flocculans | A0A318E3K1 |
| Hydrogenophaga sp. | A0A358B784 |
| Ideonella sp. KYPY4 | A0A437RLM1 |
| Alkalispirillum mobile | A0A498CBI8 |
| Hydrogenophaga sp. PAMC20947 | A0A4P7R9T3 |
| Rivibacter subsaxonicus | A0A4Q7VD40 |
| Cocleimonas flava | A0A4R1EYF4 |
| Hydrogenophaga pseudoflava | A0A4V1AB68 |

The amino acid sequences of the RcoM homologs listed above are herein incorporated by reference as they appeared in the UniProt database on May 11, 2020.

In some embodiments, the RcoM protein is from Hydrogenophaga crassostreae. In some examples, the RcoM protein has an amino acid sequence at least 80%, at least 85%, at least 90%, at least 95%, at least 96%, at least 97%, at least 98% or at least 99% identical to SEQ ID NO: 4. In some examples, the amino acid sequence of the RcoM protein comprises or consists of SEQ ID NO: 4.

Full Length RcoM Sequence from H. crassostreae

(SEQ ID NO: 4)
MEAEVANKSPLYLLEKFEVGVIHLDAKRTVLAMNDFARK
VLPVGEKQPFDKLVSSFHPARSKPKVDFLLDQASSCPMV
SAVPMTMIINIPEQVLLIKVTRLADHMGKTTGFVLVFYD
VTQVVSQEVAASEPPSTSVRLTRIPMVANHKVAFVDTQD
VLCLESQAHSTRILTRDGFHFCNLSIGDLESRLDPEQFM
RIHRCFIVNLQGVAELGREGSKTHVVLKGKNKEPVPVAR
GDVLRLRKALGLLSRH.

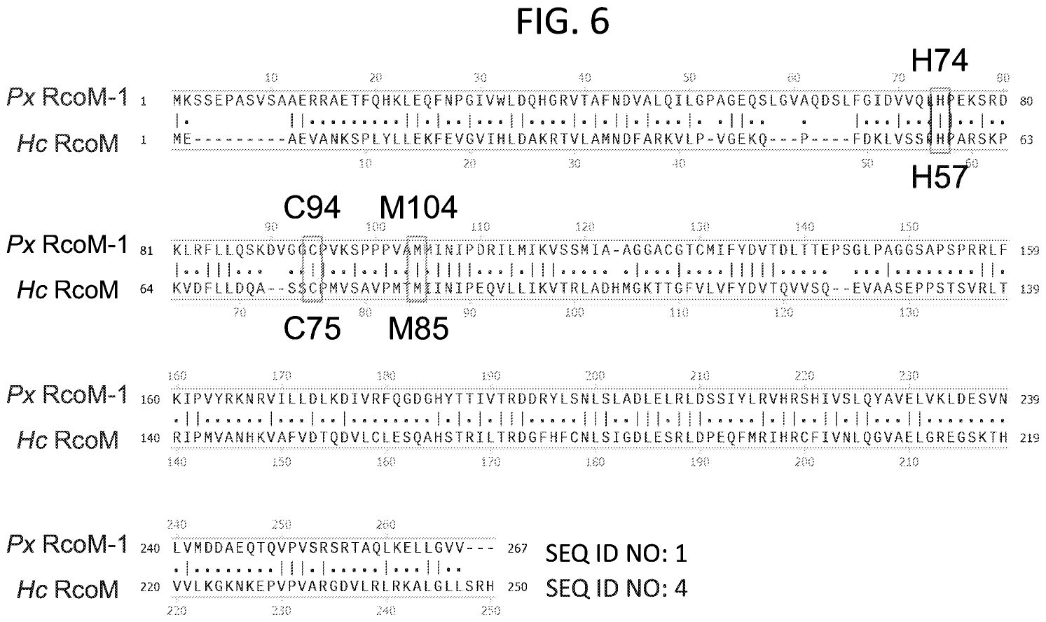

In specific non-limiting examples, the RcoM protein is at least 90% identical to SEQ ID NO: 4 and contains one or more of the amino acid substitutions described above for the RcoM-1 homolog from P. xenovorans (see FIG. 6 for the alignment).

In some embodiments, the recombinant RcoM protein includes a tag at the N-terminus, the C-terminus, or both. In some examples, the tag is as affinity tag, such as an affinity tag to aid in purification of the protein. Any suitable affinity tag can be used, such as one or more of His6, FLAG, glutathione S-transferase (GST), influenza virus hemagglutinin (HA), c-Myc, maltose-binding protein (MBP), protein A or protein G. In specific examples, the affinity tag is a His6 tag. In some examples, the affinity tag is cleavable. In specific examples, the cleavage tag includes the cleavage site from TEV having the amino acid sequence ENLYFQ[G/S] (SEQ ID NO: 5). In other specific examples, the cleavage tag includes cleavage site from thrombin having the amino acid sequence LVPRGS (SEQ ID NO: 6).

In some embodiments, the recombinant RcoM protein does not include a tag.

In some embodiments, the recombinant RcoM protein is in the oxidized form (the Fe(II), CO-bound heme in RcoM is oxidized to Fe(III)). Oxidation of RcoM can be achieved, for example, by exposure to an oxidizing agent. In some embodiments, the oxidizing agent is an oxygen-containing gas mixture, an oxygen-containing liquid mixture, a ferricyanide salt, or any combination thereof. In other embodiments, the oxidizing agent is a quinone, such as benzoquinone or napthaquinone. In some examples, an electron mediator (e.g. TMPD or crystal violet) is used in combination with an oxidizing agent in order to facilitate electron transfer. In other embodiments, oxidation of RcoM is accomplished by exposure to visible light. For example, RcoM bearing Fe(II), CO-bound heme can be exposed to white light (for example, by exposure to an incandescent bulb, such as a halogen lamp) using either an optical fiber or a heat sink screen with intensity ranging from 0.15 W/cm² to 140 W/cm² for a duration of about 1-12 hours in the presence of air. Similar methods are described in Kerby et al. (J. Bacteriol 190:3336-3343, 2008), Bouzhir-Sima et al. (J Phys Chem B 120:10686-10694, 2016) and Salman et al. (Biochem 58:4028-4034, 2019).

### V. Pharmaceutical Compositions

The recombinant RcoM proteins described herein can be administered as isolated proteins or as part of a pharmaceutical composition. Accordingly, provided herein are pharmaceutical compositions that include a recombinant RcoM disclosed herein, or a derivative thereof, and one or more pharmaceutically acceptable excipients, and optionally one or more other active (therapeutic) ingredients. The excipient(s) are “acceptable” in the sense of being compatible with the other ingredients of the formulation and not deleterious to the recipient thereof. Proper formulation of the pharmaceutical composition is dependent upon several factors, such as the route of administration chosen. Any of the well-known techniques and excipients may be used as suitable and as understood in the art. The pharmaceutical compositions disclosed herein can be manufactured in any manner known in the art, e.g., by means of conventional mixing, dissolving, granulating, dragee-making, levigating, emulsifying, encapsulating, entrapping or compression processes.

In some embodiments, disclosed are pharmaceutical compositions that include one or more recombinant RcoM proteins disclosed herein, together with one or more pharmaceutically acceptable carriers thereof and optionally one or more other therapeutic ingredients. The excipient(s)/carrier(s) must be “acceptable” in the sense of being compatible with the other ingredients of the formulation and not deleterious to the recipient thereof. Proper formulation of the pharmaceutical composition is dependent upon the route of administration chosen. Any of the well-known techniques and excipients may be used as suitable and as understood in the art. In some embodiments, the composition includes one or more of the following excipients: N-acetyl cysteine, sodium citrate, glycine, histidine, glutamic acid, sorbitol, maltose, mannitol, trehalose, lactose, glucose, raffinose, dextrose, dextran, ficoll, gelatin, hydroxyethyl starch, benzalkonium chloride, benzethonium chloride, benzyl alcohol, chlorobutanol, m-cresol, myristyl gamma-picolinium chloride, paraben methyl, paraben propyl, 2-penoxythanol, phenyl mercuric nitrate, thimerosal, acetone sodium bisulfite, argon, ascorbyl palmitate, ascorbate (sodium/acid), bisulfite sodium, butylated hydroxy anisole (BHA), butylated hydroxy toluene (BHT), cysteine/cysteinate HCl, dithionite sodium (Na hydrosulfite, Na sulfoxylate), gentisic acid, gentisic acid ethanolamine, glutamate monosodium, glutathione, formaldehyde sulfoxylate sodium, metabisulfite potassium, metabisulfite sodium, methionine, monothioglycerol (thioglycerol), nitrogen, propyl gallate, sulfite sodium, tocopherol alpha, alpha tocopherol hydrogen succinate, and thioglycolate sodium. The present disclosure also contemplates other excipients, including any disclosed in Pramanick et al., Pharma Times 45(3): 65-77, 2013, which is herein incorporated by reference.

In some embodiments, the RcoM protein of the pharmaceutical composition is pegylated, polymerized or cross-linked.

In some embodiments, the pharmaceutical composition further includes a native or recombinant globin molecule, such as a native or recombinant hemoglobin or neuroglobin, or includes a hemoglobin-based oxygen carrier (HBOC). In some examples, the HBOC includes DCLHb (HEMASSIST™; Baxter), MP4 (HEMOSPAN™; Sangart), pyridoxylated Hb POE−conjugate (PHP)+catalase & SOD (Apex Biosciences), O—R-PolyHbA₀ (HEMOLINK™; Hemosol), PolyBvHb (HEMOPURE™; Biopure), PolyHb (POLYHEME™; Northfield), rHb1.1 (OPTRO™; Somatogen), PEG-Hemoglobin (Enzon), OXYVITA™ or HBOC-201, or any combination thereof.

The pharmaceutical compositions disclosed herein can be administered by a variety of routes, depending upon whether local or systemic treatment is desired and upon the area to be treated.

The pharmaceutical compositions include those suitable for parenteral (including subcutaneous, intradermal, intramuscular, intravenous, intraarticular, and intramedullary), or intraperitoneal administration, although the most suitable route may depend upon for example the condition and disorder of the recipient. Parenteral administration includes intravenous, intraarterial, subcutaneous, intraperitoneal, intramuscular or injection or infusion; or intracranial, e.g., intrathecal or intraventricular, administration. Parenteral administration can be in the form of a single bolus dose, or may be, for example, by a continuous perfusion pump. Conventional pharmaceutical carriers, aqueous, powder or oily bases, thickeners and the like may be necessary or desirable. In some embodiments, the compounds can be contained in such pharmaceutical compositions with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The artisan can refer to various pharmacologic references for guidance. For example, Modern Pharmaceutics, 5th Edition, Banker & Rhodes, CRC Press (2009); and Goodman & Gilman's The Pharmaceutical Basis of Therapeutics, 13th Edition, McGraw Hill, New York (2018) can be consulted. The compositions can conveniently be presented in unit dosage form and may be prepared by any of the methods well known in the art of pharmacy. Typically, these methods include the step of bringing into association an isolated, recombinant RcoM molecule disclosed herein or a derivative thereof (“active ingredient”) with the carrier which constitutes one or more accessory ingredients. In general, the compositions are prepared by uniformly and intimately bringing into association the active ingredient with liquid carriers or finely divided solid carriers or both and then, if necessary, shaping the product into the desired composition.

The recombinant RcoM proteins can be formulated for parenteral administration by injection. Compositions for injection may be presented in unit dosage form, e.g., in ampoules or in multi-dose containers, with an added preservative. The pharmaceutical compositions can take such forms as suspensions, solutions or emulsions in oily or aqueous vehicles, and can contain formulatory agents such as suspending, stabilizing and/or dispersing agents. The compositions can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Pharmaceutical compositions for parenteral administration include aqueous and non-aqueous (oily) sterile injection solutions of the active compounds which can contain antioxidants, buffers, bacteriostats and solutes which render the composition isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions.

It should be understood that in addition to the ingredients particularly mentioned above, the pharmaceutical compositions described above can include other agents conventional in the art having regard to the type of pharmaceutical composition in question, for example those suitable for oral administration can include flavoring agents.

Unit dosage pharmaceutical compositions are those containing an effective dose, as hereinbelow recited, or an appropriate fraction thereof, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The RcoM proteins can be effective over a wide dosage range and can be generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the disclosed recombinant RcoM proteins can be administered at a therapeutically effective dose of from about 0.01 g to about 1000 g per day. In some examples, the dose of recombinant RcoM proteins is about 0.1 g to about 900 g, about 0.1 g to about 800 g, about 0.1 g to about 700 g, about 0.1 g to about 600 g, about 0.1 g to about 500 g, about 0.1 g to about 400 g, about 0.1 g to about 300 g, about 0.1 g to about 200 g, about 0.1 g to about 100 g, about 1 g to about 900, about 1 g to about 800, about 1 g to about 700 g, about 1 g to about 600, about 1 g to about 500, about 1 g to about 400, about 1 g to about 300 g, about 1 g to about 200 g, about 1 g to about 100 g, about 10 g to about 900, about 10 g to about 800 g, about 10 g to about 700 g, about 10 g to about 600 g, about 10 g to about 500 g, about 10 g to about 400 g, about 10 g to about 300 g, about 10 g to about 200 g, or about 10 g to about 100 g, or a range between any two of these values.

The amount of active ingredient that is combined with the carrier materials to produce a single dosage form will vary depending upon the host treated and the particular mode of administration. In some embodiments disclosed herein, the pharmaceutical compositions include one or more of the disclosed RcoM proteins (as the active ingredient) in combination with one or more pharmaceutically acceptable carriers (excipients).

In some embodiments, the one or more recombinant RcoM protein constitute about 0.01% to about 50% of the pharmaceutical composition. In some embodiments, the one or more RcoM proteins constitute about 0.01% to about 50%, about 0.01% to about 45%, about 0.01% to about 40%, about 0.01% to about 30%, about 0.01% to about 20%, about 0.01% to about 10%, about 0.01% to about 5%, about 0.05% to about 50%, about 0.05% to about 45%, about 0.05% to about 40%, about 0.05% to about 30%, about 0.05% to about 20%, about 0.05% to about 10%, about 0.1% to about 50%, about 0.1% to about 45%, about 0.1% to about 40%, about 0.1% to about 30%, about 0.1% to about 20%, about 0.1% to about 10%, about 0.1% to about 5%, about 0.5% to about 50%, about 0.5% to about 45%, about 0.5% to about 40%, about 0.5% to about 30%, about 0.5% to about 20%, about 0.5% to about 10%, about 0.5% to about 5%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 5% to about 45%, about 5% to about 40%, about 5% to about 35%, about 5% to about 30%, about 5% to about 25%, about 5% to about 20%, about 5% to about 15%, about 5% to about 10%, about 10% to about 45%, about 10% to about 40%, about 10% to about 35%, about 10% to about 30%, about 10% to about 25%, about 10% to about 20%, about 10% to about 15%, or a value within one of these ranges. Specific non-limiting examples include about 0.01%, about 0.05%, about 0.1%, about 0.25%, about 0.5%, about 0.75%, about 1%, about 5%, about 10%, about 15%, about 20%, about 25%, about 30%, about 35%, about 40%, about 45%, about 50%, about 60%, about 70%, about 80%, about 90%, or a range between any two of these values. The foregoing all representing weight percentages of the pharmaceutical composition.

The amount of recombinant RcoM protein administered to a patient will vary depending upon what is being administered, the purpose of the administration, such as prophylaxis or therapy, the state of the patient, the manner of administration, and the like. In therapeutic applications, compositions can be administered to a patient already suffering from a disease or condition in an amount sufficient to cure or at least partially arrest the symptoms of the disease and its complications.

In some embodiments, the pharmaceutical compositions can be sterilized by conventional sterilization techniques, or may be sterile filtered. Aqueous solutions can be packaged for use as is, or lyophilized, the lyophilized preparation being combined with a sterile aqueous carrier prior to administration. In some embodiments, the pH of the RcoM protein preparations is about 3 to about 11, about 5 to about 9, about 5.5 to about 6.5, or about 5.5 to about 7.5. It will be understood that use of certain of the foregoing excipients, carriers, or stabilizers will result in the formation of pharmaceutical salts.

In certain embodiments, the pharmaceutical composition includes a reducing agent. In some examples, the reducing agent is selected from ascorbic acid, N-acetylcysteine, sodium dithionite, methylene blue, glutathione, B5/B5-reductase/NADH, tris(2-carboxyethyl)phosphine, dithiothreitol, or a combination thereof. Other agents with the property to reduce an iron containing heme molecule could also be used.

In other particular embodiments, the pharmaceutical composition includes an oxidizing agent. In some examples, the oxidizing agent is selected from an oxygen-containing gas mixture, an oxygen-containing liquid mixture, a ferricyanide salt, or any combination thereof.

In certain embodiments, the pharmaceutical compositions can be de-oxygenated by producing and maintaining the RcoM proteins or pharmaceutical composition in an oxygen-free environment.

### VI. Methods of Treating CO, H₂S and Cyanide Poisoning

The recombinant RcoM proteins disclosed herein (see Section IV) exhibit extraordinarily high affinity for carbon monoxide. Based on this property, the disclosed RcoM proteins can be used in a variety of in vivo and in vitro methods, including as an antidote for carbon monoxide poisoning. Use of the disclosed RcoM proteins for treating cyanide and hydrogen sulfide (H₂S) poisoning is also described.

Provided herein are methods of treating carboxyhemoglobinemia (carbon monoxide poisoning) in a subject. In some embodiments, the method includes administering to the subject a therapeutically effective amount of a recombinant RcoM protein disclosed herein, or a pharmaceutical composition containing a recombinant RcoM protein. In some embodiments, the method includes selecting a subject with carboxyhemoglobinemia (carbon monoxide poisoning) prior to administration of the RcoM protein or pharmaceutical composition thereof. In some examples, the subject has at least 3%, at least 5%, at least 10%, at least 15%, at least 20%, at least 30%, at least 40% or at least 50% carboxyhemoglobin in their blood. In some embodiments, the RcoM protein is in its reduced form. In some examples, the reducing agent includes sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), trehalose, reducing carbohydrate (such as sorbitol or mannitol), or any combination thereof.

Further provided herein is a method of removing carbon monoxide from native hemoglobin, myoglobin or mitochondria (i.e. from cytochrome c oxidase in mitochondria) in blood or tissue of a subject, by contacting the subject's blood or tissue with a recombinant RcoM protein or pharmaceutical composition disclosed herein. In some embodiments, the method includes selecting a subject with carboxyhemoglobinemia (carbon monoxide poisoning) prior to contacting the subject's blood or tissue with a disclosed RcoM protein or pharmaceutical composition thereof. In some examples, the subject has at least 5%, at least 10%, at least 15%, at least 20%, at least 30%, at least 40% or at least 50% carboxyhemoglobin in their blood. In some embodiments, the RcoM protein is in its reduced form. In some examples, the reducing agent includes sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), trehalose, reducing carbohydrate (such as sorbitol or mannitol), or any combination thereof.

Also provided herein is a method of removing hydrogen sulfide from native hemoglobin, myoglobin or mitochondria (such as from cytochrome c oxidase in mitochondria) in blood or tissue of a subject, by contacting the subject's blood or tissue with a recombinant RcoM protein or pharmaceutical composition disclosed herein. In some examples, the method further include the step of selecting a subject with hydrogen sulfide poisoning prior to contacting the subject's blood or tissue with the RcoM protein or pharmaceutical composition. A method of treating hydrogen sulfide poisoning in a subject by administering to the subject a therapeutically effective amount of a recombinant RcoM protein or pharmaceutical compositions disclosed herein is further provided. In some examples, the method further include the step of selecting a subject with hydrogen sulfide poisoning prior to administering the RcoM protein or pharmaceutical composition. In some embodiments of these methods, the RcoM protein is in its reduced form. Examples of reducing agents to include in the pharmaceutical composition include, but are not limited to, sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), trehalose, reducing carbohydrate (such as sorbitol or mannitol), or any combination thereof.

Further provided herein is a method of removing cyanide from native hemoglobin, myoglobin or mitochondria (such as from cytochrome c oxidase in mitochondria) in blood or tissue of a subject, by contacting the subject's blood or tissue with a recombinant RcoM protein or pharmaceutical composition disclosed herein. In some examples, the method further include the step of selecting a subject with cyanide poisoning prior to contacting the subject's blood or tissue with the RcoM protein or pharmaceutical composition. A method of treating cyanide poisoning in a subject by administering to the subject a therapeutically effective amount of a recombinant RcoM protein or pharmaceutical compositions disclosed herein is also provided. In some examples, the method further include the step of selecting a subject with cyanide poisoning prior to administering the RcoM protein or pharmaceutical composition. In some embodiments of these methods, the RcoM protein is in its oxidized form. In some examples, the oxidizing agent includes an oxygen-containing gas mixture, an oxygen-containing liquid mixture, a ferricyanide salt, or any combination thereof.

In some embodiments of the in vivo methods disclosed herein, the RcoM protein or pharmaceutical composition is administered intravenously or intramuscularly. In some examples, the RcoM protein or pharmaceutical composition is administered by intravenous infusion, intraperitoneal injection or intramuscular injection.

In some embodiments, the RcoM protein is administered, either alone or as part of a pharmaceutical composition, at a dose of about 0.1 to about 300 g per day. Additional dose ranges are described above in Section V.

Also provided herein is an in vitro method of removing carbon monoxide from hemoglobin, myoglobin or mitochondria (e.g. from cytochrome c oxidase in mitochondria) in blood or animal tissue, comprising contacting the blood or animal tissue with an effective amount of a recombinant RcoM protein disclosed herein. In some embodiments, the RcoM protein is in its reduced form.

Further provided herein is an in vitro method of removing hydrogen sulfide from hemoglobin, myoglobin or mitochondria (e.g. from cytochrome c oxidase in mitochondria) in blood or animal tissue, comprising contacting the blood or animal tissue with an effective amount of a recombinant RcoM protein disclosed herein. In some embodiments, the RcoM protein is in its reduced form.

Also provided herein is an in vitro method of removing cyanide from hemoglobin, myoglobin or mitochondria (e.g. from cytochrome c oxidase in mitochondria) in blood or animal tissue, comprising contacting the blood or animal tissue with an effective amount of a recombinant RcoM protein disclosed herein. In some embodiments, the RcoM protein is in its oxidized form.

In some embodiments of the disclosed methods, the recombinant RcoM protein is pegylated, polymerized or cross-linked.

### VII. Recombinant RcoM as a Blood Substitute

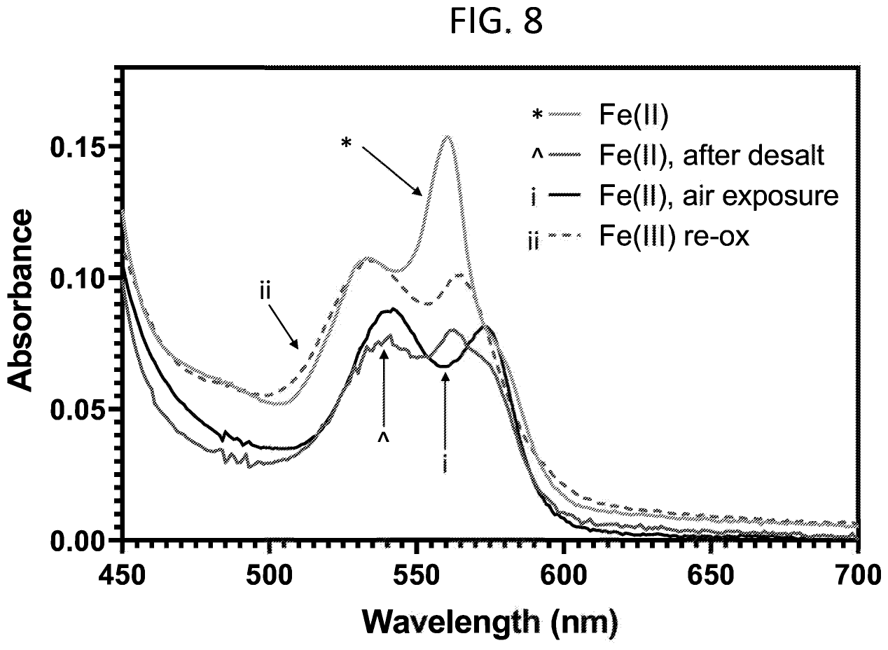

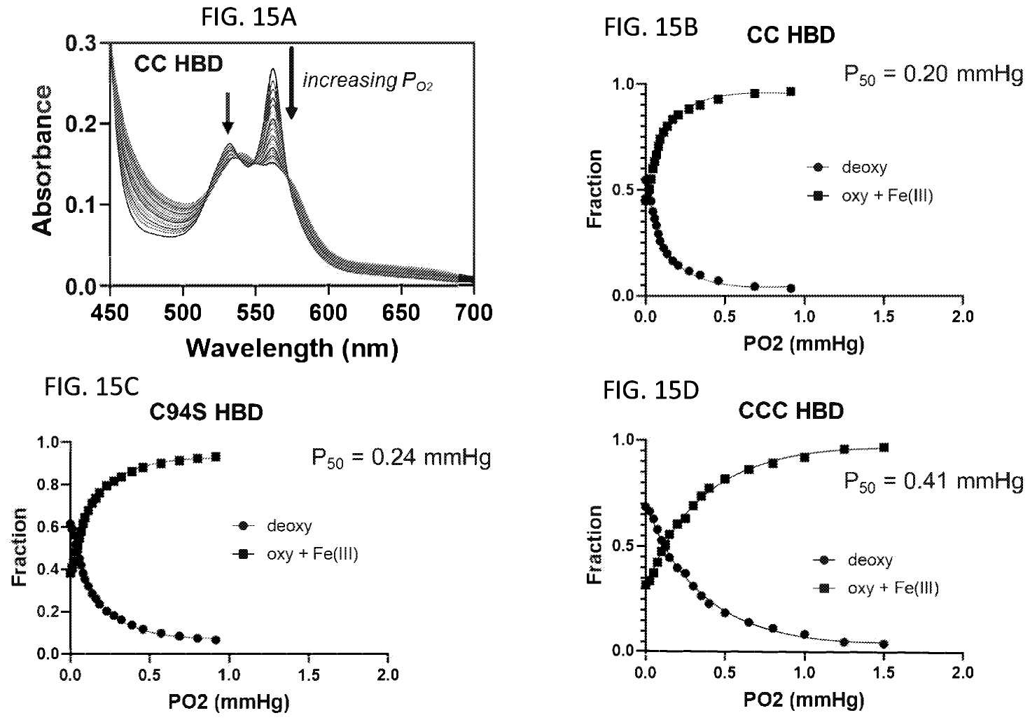

The recombinant RcoM proteins disclosed herein are capable of binding and carrying oxygen (see FIGS. 8 and 15A-15D; Examples 3 and 4). Thus, use of the disclosed RcoM proteins as blood substitutes is contemplated.

Provided herein is a method of replacing blood and/or increasing oxygen delivery to tissues in a subject. In some embodiments, the method includes administering to the subject a therapeutically effective amount of a recombinant RcoM protein or pharmaceutical composition disclosed herein, thereby replacing blood and/or increasing oxygen delivery in the subject.

The subject to be treated, for example, is any subject in need of increasing blood volume or increasing oxygen delivery to tissues. In some embodiments, the subject has or is at risk of developing a disease, disorder or injury associated with a deficiency in red blood cells and/or hemoglobin, or associated with a reduction in oxygen delivery to tissues. In some examples, the disease, disorder or injury comprises a bleeding disorder, a bleeding episode, anemia, shock, ischemia, hypoxia, anoxia, hypoxemia, a burn, an ulcer, ectopic pregnancy, microcytosis, rhabdomyolysis, hemoglobinopathy, spherocytosis, hemolytic uremic syndrome, thalassemia, disseminating intravascular coagulation, stroke or yellow fever.

In some embodiments, the bleeding episode in the subject to be treated with a recombinant RcoM protein results from anticoagulant overdose, aneurysm, blood vessel rupture, surgery, traumatic injury, gastrointestinal bleeding, pregnancy, hemorrhage or infection.

In some embodiments, the bleeding disorder in the subject to be treated with a recombinant RcoM protein comprises hemophilia A, hemophilia B, hemophilia C, Factor VII deficiency, Factor XIII deficiency, a platelet disorder, a coagulopathy, favism, thrombocytopenia, vitamin K deficiency or von Willebrand's disease.

In some embodiments, the anemia in the subject to be treated comprises microcytic anemia, iron deficiency anemia, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle-cell anemia, warm autoimmune hemolytic anemia or cold agglutinin hemolytic anemia.

In some embodiments, shock in the subject to be treated with comprises septic shock, hemorrhagic shock or hypovolemic shock.

In some embodiments, the subject to be treated suffers from or is at risk of suffering from a disease or condition associated with decreased blood flow, such that increased oxygen delivery is beneficial for treatment of the subject. Examples of diseases or conditions that can be treated using the disclosed methods include, but are not limited to, ischemia, myocardial infarction, stroke, ischemia-reperfusion injury, elevated blood pressure, pulmonary hypertension (including neonatal pulmonary hypertension, primary pulmonary hypertension, and secondary pulmonary hypertension), systemic hypertension, cutaneous ulceration, acute renal failure, chronic renal failure, intravascular thrombosis, an ischemic central nervous system event, vasospasm (such as cerebral artery vasospasm), a hemolytic condition, peripheral vascular disease, trauma, cardiac arrest, general surgery or organ transplantation.

In some embodiments, the recombinant RcoM protein is administered to the subject intravenously.

In some embodiments, the method further includes administering to the subject a second blood replacement product, a blood product or whole blood. In some examples, the second blood replacement product comprises a hemoglobin-based oxygen carrier, artificial red blood cells or an oxygen releasing compound. In some examples, the blood product comprises packed red blood cells, plasma or serum.

In some examples, the subject is a human. In other examples, the subject is a non-human animal.

Also provided are compositions that include a disclosed RcoM protein and an oxygen carrier, such as a native or recombinant globin molecule (such as a native or recombinant hemoglobin or neuroglobin), or a hemoglobin-based oxygen carrier (HBOC). In some embodiments, the composition further includes a pharmaceutically acceptable carrier or excipient, or both. In some examples, the RcoM protein in the composition is pegylated, polymerized or cross-linked.

### VIII. Embodiments

Embodiment 1. A recombinant regulator of carbon monoxide metabolism (RcoM) protein, wherein the recombinant RcoM protein comprises a heme-binding domain (HBD), and wherein the amino acid sequence of the HBD is at least 90% identical to SEQ ID NO: 2 and comprises an amino acid substitution at one or more of H74, C94, M104, M105, C127 and C130.

Embodiment 2. The recombinant RcoM protein of embodiment 1, wherein:

the substitution at H74 is selected from H74S, H74T, H74M, H74W, H74A, H74L, H74I, H74V and H74G;

the substitution at C94 is selected from C94S, C94T, C94H, C94W, C94M, C94A, C94L, C94I, C94V and C94G;

the substitution at M104 is selected from M104S, M104T, M104H, M104W, M104A, M104L, M104I, M104V and M104G;

the substitution at M105 is selected from M105S, M105T, M105H, M105W, M105A, M105L, M105I, M105V and M105G;

the substitution at C127 is selected from C127S, C127T, C127M, C127A, C127L, C127I, C127V and C127G; and/or

the substitution at C130 is selected from C130S, C130T, C130M, C130A, C130L, C130I, C130V and C130G.

Embodiment 3. The recombinant RcoM protein of embodiment 1 or embodiment 2, wherein the amino acid sequence of the HBD is at least 95% identical to SEQ ID NO: 2 and comprises an amino acid substitution at one or more of C94, M104, C127 and C130.

Embodiment 4. The recombinant RcoM protein of any one of embodiments 1-4, wherein the HBD comprises:

a C94S substitution;

a C127S substitution and a C130S substitution;

a C94S substitution, a C127S substitution and a C130S substitution;

a M104A substitution, a C127S substitution and a C130S substitution;

a M104H substitution, a C127S substitution and a C130S substitution;

a M104L substitution, a C127S substitution and a C130S substitution;

a C94S substitution, a M104A substitution, a C127S substitution and a C130S substitution;

a C94S substitution, a M104H substitution, a C127S substitution and a C130S substitution; or

a C94S substitution, a M104L substitution, a C127S substitution and a C130S substitution.

Embodiment 5. The recombinant RcoM protein of any one of embodiments 1-4, wherein:

the amino acid sequence of the RcoM protein comprises or consists of SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or

the amino acid sequence of the RcoM protein comprises or consists of SEQ ID NO: 1 or SEQ ID NO: 2, except for an amino acid substitution at one or more of H74, C94, M104, C127, C130 and M105.

Embodiment 6. The recombinant RcoM protein of any one of embodiments 1-5, wherein the RcoM protein comprises an N-terminal tag or a C-terminal tag.

Embodiment 7. The recombinant RcoM protein of embodiment 6, wherein the tag is an affinity tag.

Embodiment 8. The recombinant RcoM protein of embodiment 7, wherein the affinity tag is His6, FLAG, glutathione S-transferase (GST), influenza virus hemagglutinin (HA), c-Myc, maltose-binding protein (MBP), protein A or protein G.

Embodiment 9. The recombinant RcoM protein of any one of embodiments 6-8, wherein the tag is cleavable.

Embodiment 10. An in vitro method of removing carbon monoxide from hemoglobin, myoglobin or mitochondria in blood or animal tissue, comprising contacting the blood or animal tissue with an effective amount of the recombinant RcoM protein of any one of embodiments 1-9, thereby removing carbon monoxide from hemoglobin in the blood or animal tissue.

Embodiment 11. A method of treating carboxyhemoglobinemia in a subject, comprising administering to the subject a therapeutically effective amount of the RcoM protein of any one of embodiments 1-9.

Embodiment 12. The method of embodiment 11, further comprising selecting a subject with carboxyhemoglobinemia prior to administering the recombinant RcoM protein.

Embodiment 13. The method of embodiment 11 or embodiment 12, wherein the subject has at least 5%, at least 10%, at least 15%, at least 20%, at least 30%, at least 40% or at least 50% carboxyhemoglobin in their blood.

Embodiment 14. The method of any one of embodiments 11-13, wherein the recombinant RcoM protein is administered by intravenous infusion, intraperitoneal injection or intramuscular injection.

Embodiment 15. The method of any one of embodiments 11-14, wherein the recombinant RcoM protein is administered at a dose of about 0.1 g to about 300 g per day.

Embodiment 16. The method of any one of embodiments 11-15, wherein the recombinant RcoM protein is administered as a pharmaceutical composition comprising a reducing agent.

Embodiment 17. The method of embodiment 16, wherein the reducing agent comprises sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), or any combination thereof.

Embodiment 18. A method of treating cyanide poisoning in a subject, comprising administering to the subject a therapeutically effective amount of the recombinant RcoM protein of any one of embodiments 1-9, wherein the RcoM protein is in its oxidized form, thereby treating cyanide poisoning in the subject.

Embodiment 19. The method of embodiment 18, further comprising selecting a subject with cyanide poisoning prior to administering the recombinant RcoM protein.

Embodiment 20. The method of embodiment 18 or embodiment 19, wherein the recombinant RcoM protein is administered as a pharmaceutical composition comprising an oxidizing agent.

Embodiment 21. The method of embodiment 20, wherein the oxidizing agent comprises an oxygen-containing gas mixture, an oxygen-containing liquid mixture, a ferricyanide salt, or any combination thereof.

Embodiment 22. A method of treating hydrogen sulfide (H₂S) poisoning in a subject, comprising administering to the subject a therapeutically effective amount of the recombinant RcoM protein of any one of embodiments 1-9, wherein the RcoM protein is in its reduced form, thereby treating H2S poisoning in the subject.

Embodiment 23. The method of embodiment 22, further comprising selecting a subject with H2S poisoning prior to administering the recombinant RcoM protein.

Embodiment 24. The method of embodiment 22 or embodiment 23, wherein the recombinant RcoM protein is administered as a pharmaceutical composition comprising a reducing agent.

Embodiment 25. The method of embodiment 24, wherein the reducing agent comprises sodium dithionite, ascorbic acid, N-acetylcysteine (NAC), methylene blue, glutathione, cytochrome b5/b5-reductase, hydralazine, tris(2-carboxyethyl)phosphine (TCEP), trehalose, dithiothreitol (DTT), or any combination thereof.

Embodiment 26. A method of replacing blood in a subject, comprising administering to the subject a therapeutically effective amount of the recombinant RcoM protein of any one of embodiments 1-9, thereby replacing blood in the subject.

Embodiment 27. The method of embodiment 26, wherein the subject has or is at risk of developing a disease, disorder or injury associated with a deficiency in red blood cells and/or hemoglobin, or associated with a reduction in oxygen delivery to tissues.

Embodiment 28. The method of embodiment 27, wherein the disease, disorder or injury comprises a bleeding disorder, a bleeding episode, anemia, shock, ischemia, hypoxia, anoxia, hypoxaemia, a burn, an ulcer, ectopic pregnancy, microcytosis, rhabdomyolysis, hemoglobinopathy, spherocytosis, hemolytic uremic syndrome, thalassemia, disseminating intravascular coagulation, stroke or yellow fever.

Embodiment 29. The method of embodiment 28, wherein:

the bleeding episode results from anticoagulant overdose, aneurysm, blood vessel rupture, surgery, traumatic injury, gastrointestinal bleeding, pregnancy, hemorrhage or infection;

the bleeding disorder comprises hemophilia A, hemophilia B, hemophilia C, Factor VII deficiency, Factor XIII deficiency, a platelet disorder, a coagulopathy, favism, thrombocytopenia, vitamin K deficiency or von Willebrand's disease;

the anemia comprises microcytic anemia, iron deficiency anemia, heme synthesis defect, globin synthesis defect, sideroblastic defect, normocytic anemia, anemia of chronic disease, aplastic anemia, hemolytic anemia, macrocytic anemia, megaloblastic anemia, pernicious anemia, dimorphic anemia, anemia of prematurity, Fanconi anemia, hereditary spherocytosis, sickle-cell anemia, warm autoimmune hemolytic anemia or cold agglutinin hemolytic anemia; or

shock comprises septic shock, hemorrhagic shock or hypovolemic shock.

Embodiment 30. The method of embodiment 26, wherein the subject suffers from or is at risk of suffering from myocardial infarction, stroke, ischemia-reperfusion injury, pulmonary hypertension or vasospasm.

Embodiment 31. The method of any one of embodiments 26-30, wherein the recombinant RcoM protein is administered to the subject intravenously.

Embodiment 32. The embodiment of any one of claims 26-31, wherein the recombinant RcoM protein is pegylated, polymerized or cross-linked.

Embodiment 33. The method of any one of embodiments 26-32, further comprising administering to the subject a second blood replacement product, a blood product or whole blood.

Embodiment 34. The method of embodiment 33, wherein the second blood replacement product comprises a hemoglobin-based oxygen carrier, artificial red blood cells or an oxygen releasing compound.

Embodiment 35. The method of embodiment 33, wherein the blood product comprises packed red blood cells, plasma or serum.

Embodiment 36. The method of any one of embodiments 11-35, wherein the subject is a human

Embodiment 37. The method of any one of embodiments 11-35, wherein the subject is a non-human animal.

Embodiment 38. A pharmaceutical composition, comprising the recombinant RcoM protein of any one of embodiments 1-9 and a pharmaceutically acceptable carrier.

Embodiment 39. The pharmaceutical composition of embodiment 38, further comprising a reducing agent or an oxidizing agent.

Embodiment 40. The pharmaceutical composition of embodiment 39, wherein the reducing agent comprises sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), or any combination thereof.

Embodiment 41. The pharmaceutical composition of embodiment 39, wherein the oxidizing agent comprises an oxygen-containing gas mixture, an oxygen-containing liquid mixture, a ferricyanide salt, a quinone, or any combination thereof.

Embodiment 42. The pharmaceutical composition of any one of embodiments 38-41, wherein the recombinant RcoM protein is pegylated, polymerized or cross-linked.

### EXAMPLES

### Example 1: Transfer of CO from HbCO to RcoM-1 in an Aerobic Environment

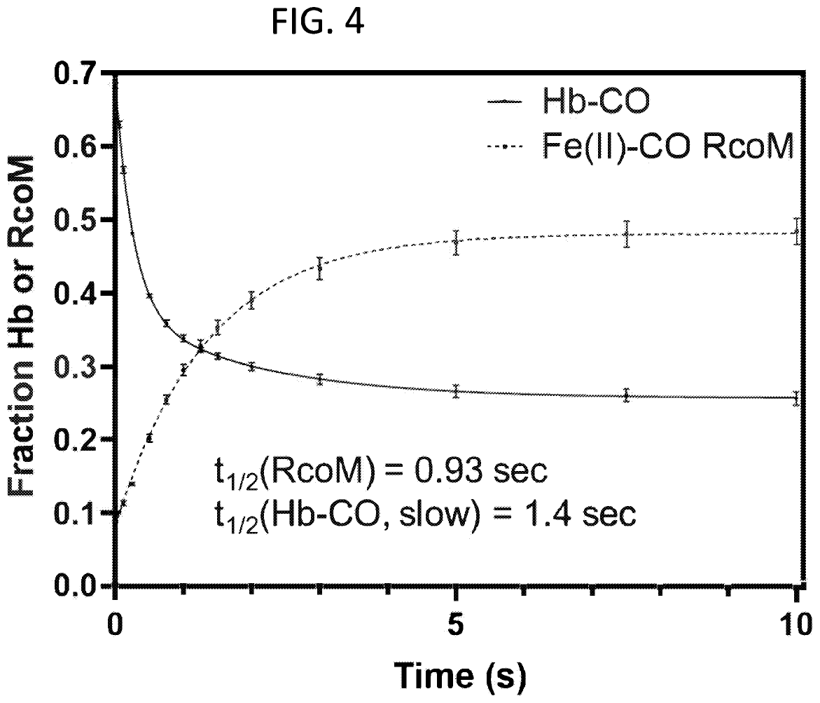

Hemoglobin-CO (Hb-CO) transfer kinetics was evaluated in the presence of WT, full-length RcoM-1 (SEQ ID NO: 1) under aerobic conditions at 37° C., and measured using stopped-flow UV-Vis spectroscopy and standard deconvolution methods based on extinction coefficients for different ligand-bound species of RcoM-1 and hemoglobin. Concentrations of Hb-CO and Fe(II) RcoM-1 were 20 μM, and experiments were performed in triplicate. The data for loss of hemoglobin-CO was fit to a double exponential curve, which exhibited a slow-phase half-life (t_{1/2}) of 1.4 seconds. The data for increase of Fe(II)-CO RcoM was fit to a single exponential curve, which exhibited a half-life of 0.93 seconds. The results are shown in FIG. 4. This data demonstrates that RcoM has as high affinity for CO and allows for rapid and efficient transfer of CO from Hb to RcoM.

### Example 2: Transfer of CO from Hb-CO to RcoM-1 in an Anaerobic Environment

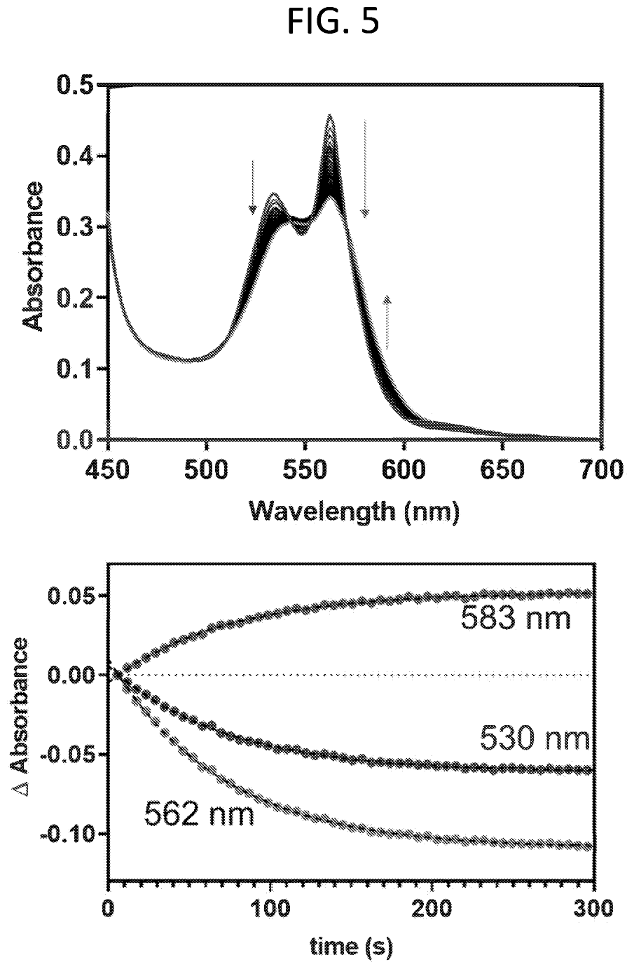

Hemoglobin-CO transfer kinetics in the presence of WT, full-length RcoM-1 (SEQ ID NO: 1) under anaerobic conditions at 37° C. was measured using UV-Vis spectroscopy. Concentrations of Hb-CO and Fe(II) RcoM-1 were 15 μM and 15.8 μM, respectively. Changes in absorbance at 530, 562, and 583 nm, which track THE transition from Fe(II) to Fe(II)-CO RcoM, were fit to a single exponential curve, which exhibited a half-life of 50 seconds. The results are shown in FIG. 5. These results demonstrate that RcoM-1 is capable of scavenging CO from the Hb-CO species, and can therefore be used as a CO scavenger in vivo.

### Example 3: Characterization of Truncated RcoM with a C94C Substitution (HBD C94S)

This example describes studies to characterize a modified RcoM protein lacking the PAS domain and having a C94S substitution (SEQ ID NO: 7). The results of these studies demonstrate that altering heme binding residues can alter the gas binding properties of RcoM.

### Stability

The HBD C94S mutant is much more stable than WT RcoM-1. This mutant form of RcoM can be stored at higher concentrations than WT RcoM (˜480 mM heme compared to ˜130 mM for WT RcoM). HBD C94S can also be reduced using dithionite without the presence of stabilizing reducing agents (e.g. DTT, TCEP). In addition, oxidized and reduced forms of RcoM are stable towards aggregation when stored at 4° C. for more than 1 week. Studies demonstrated that the Tₘ for Fe(III) RcoM-1 HBD C94S is 90° C. (recorded at a RcoM concentration of 7 μM under anaerobic conditions in a septum-sealed cuvette). Under the same conditions, WT RcoM-1 irreversibly unfolded at about 40° C.

### Comparison of UV-Vis Spectra

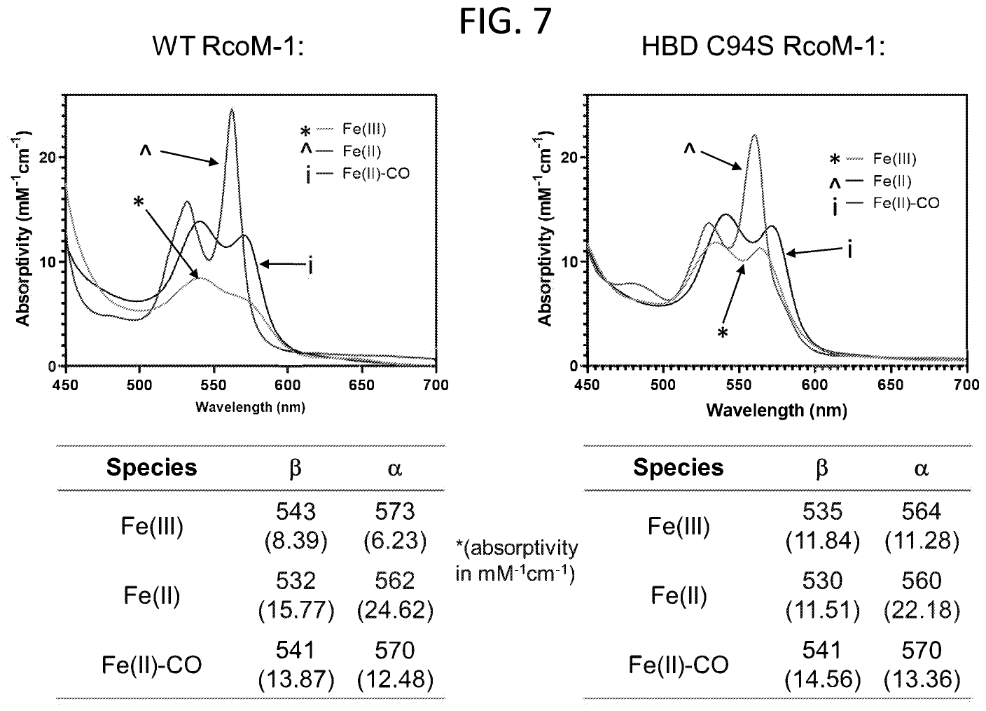

Spectra for full-length wild-type RcoM-1 and HBD C94S RcoM were evaluated. Spectra for the ferric (Fe(III)), ferrous deoxy (Fe(II)) and ferrous-CO species (Fe(II)-CO) were determined. The wavelength for the peak maxima for each species (in nm) were calculated, along with the estimated molar absorptivity for each peak (mM⁻¹cm⁻¹). The results are shown in FIG. 7. As expected, the Fe(II) and Fe(II)-CO spectra were very similar between the WT and HBD C94S RcoM proteins. However, the Fe(III) spectra looked very different, demonstrating that the Fe(III) heme coordination environment changed as a result of the C94S substitution.

An additional study provided evidence for a stable O₂ adduct in HBD C94S. HBD C94S was reduced with excess dithionite, producing the ferrous Fe(II) species. The reduced HBD C94S was then de-salted and a UV-Vis sample was prepared under micro-aerobic conditions. After recording the UV-Vis spectrum under micro-aerobic conditions, the cuvette cap was removed to introduce air, and the spectrum was re-recorded, revealing oxygen-bound species (RcoM concentration=8 μM). FIG. 8 shows the visible spectra for the ferrous (Fe(II)) species in the presence of the reductant, Fe(II) species after desalting, Fe(II) species after air exposure and re-oxidized Fe(III) species.

### HBD C94S RcoM-CO Association and Dissociation Rates

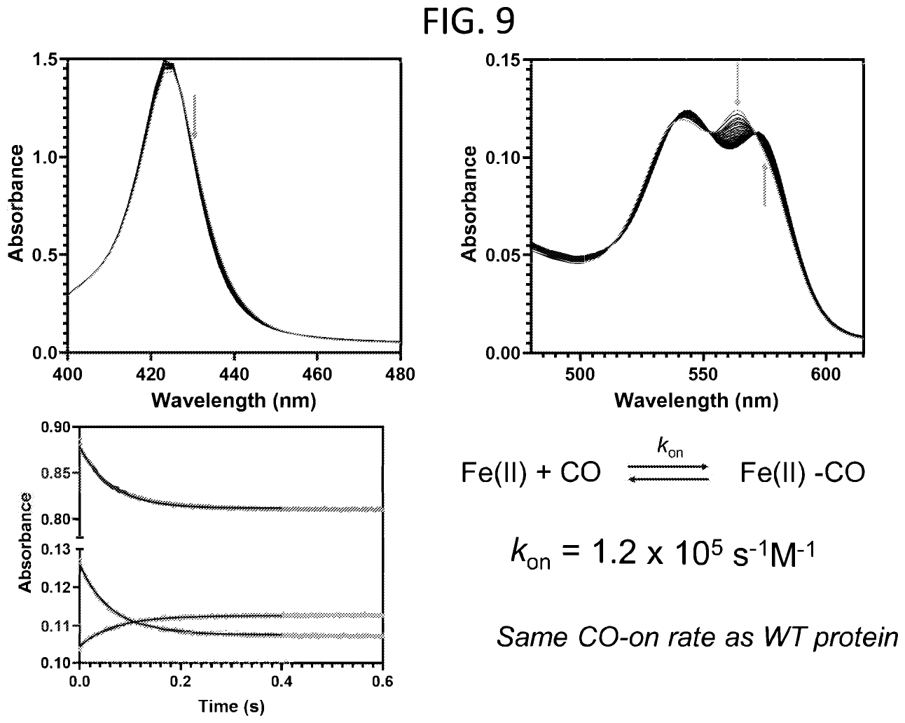

Kinetics of the reaction of the ferrous heme binding domain (HBD) of HBD C94S RcoM with carbon monoxide (CO) was determined by stopped-flow techniques (FIG. 9). The study was carried out at a RcoM concentration of 10 μM, CO concentrations of 55-287 μM, and a temperature of 37° C. The calculation of the rates at different CO concentrations yielded an association rate (kₒₙ) for the reaction of 1.2×10⁵ M⁻¹s⁻¹. Similar values were obtained for the wild-type, full length protein. Thus, the CO on rate was not affected by the C94S substitution.

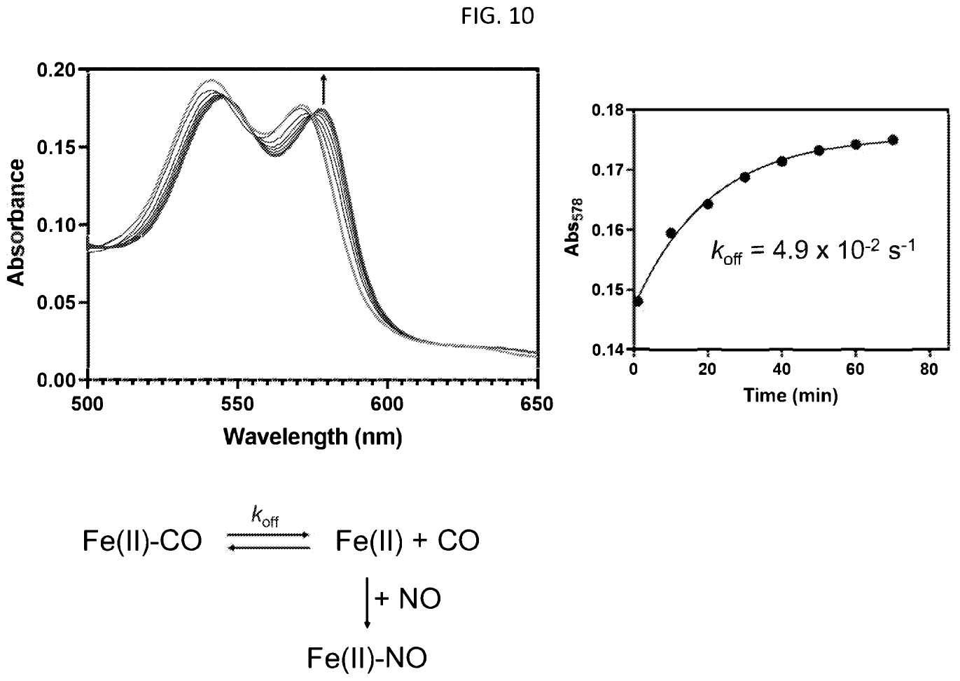

The CO dissociation rate for HBD C94S was determined using a RcoM concentration of 10 μM, nitric oxide (NO) concentration of 2 mM (generated using 1 mM ProliNONOate) and a temperature of 37° C. The reaction was monitored by the absorbance change as the ferrous-CO complex dissociates in the presence of NO. As CO dissociates, NO binds to the heme causing a change in the absorbance spectrum. Excess NO prevents CO from rebinding the heme. The time course of the absorbance changes allowed for determination of a dissociation rate of 4.9×10⁻² s⁻¹ (FIG. 10).

### Thermal Unfolding of HBD C94S

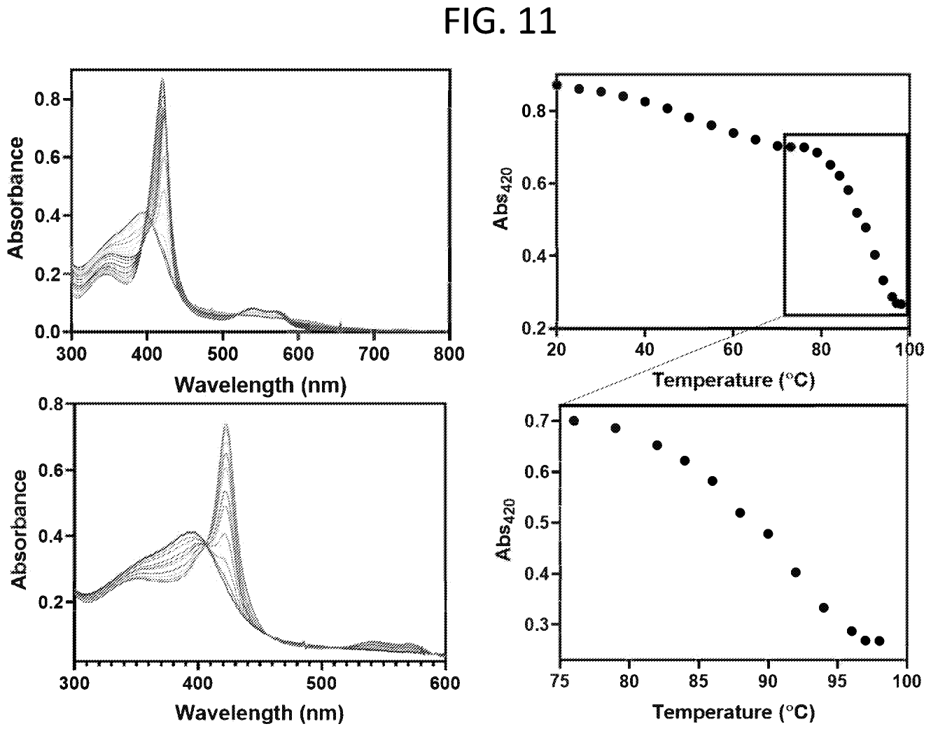

Unfolding was monitored by the change in absorbance at the heme Soret maximum of 420 nm (FIG. 11). The sample was allowed to equilibrate at each temperature for five minutes before recording each UV-Vis spectrum. A small loss in Soret intensity, observed between 20° C. and 75° C., was likely due to a change in heme coordination number. Loss of Soret intensity between 75° C. and 98° C. was attributed to loss of heme from the protein due to thermal unfolding. The UV-Vis spectra for Fe(III) HBD RcoM-1 bearing the C94S mutation was recorded at each temperature between 20° C. and 98° C. The Tₘ of HBD C94S was determined to be 91° C.

### Example 4: RcoM Heme-Binding Domain (HBD) Variants

This example describes the generation and characterization of several truncated RcoM HBD variants.

Eight RcoM variants were generated. The variants listed in Table 4 were successfully cloned, expressed in E. coli, and purified to homogeneity. The variants encompass the heme-binding domain (HBD) of RcoM-1 from Paraburkholderia xenovorans and possess various mutations (at one or more of residues C94, M104, C127, and C130) to enhance solubility, stability, and CO scavenging properties. The expressed variants also included a C-terminal 6-His tag. With the 6-His tag, the variants were 17 kDa.

TABLE 4
RcoM HDB16 variants

| | | SEQ ID | |
|---|---|---|---|
| Variant | Substitutions | NO: | Sequence (without the 6-His tag) |
| C94S HBD | Cys94→Ser | 7 | MKSSEPASVSAAERRAETFQHKLEQFNPGIV |
| | | | WLDQHGRVTAFNDVALQILGPAGEQSLGVA |
| | | | QDSLFGIDVVQLHPEKSRDKLRFLLQSKDVG |
| | | | GSPVKSPPPVAMMINIPDRILMIKVSSMIAAG |
| | | | GACGTCMIFYDVTDLTTEPSGLPAGGSAPS |
| C127S/C130S | Cys127→Ser | 8 | MKSSEPASVSAAERRAETFQHKLEQFNPGIV |
| HBD | Cys130→Ser | | WLDQHGRVTAFNDVALQILGPAGEQSLGVA |
| | | | QDSLFGIDVVQLHPEKSRDKLRFLLQSKDVG |
| | | | GCPVKSPPPVAMMINIPDRILMIKVSSMIAAG |
| | | | GASGTSMIFYDVTDLTTEPSGLPAGGSAPS |
| CCC HBD | Cys94→Ser | 9 | MKSSEPASVSAAERRAETFQHKLEQFNPGIV |
| | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GSPVKSPPPVAMMINIPDRILMIKVSSMIAAG |
| | | | GASGTSMIFYDVTDLTTEPSGLPAGGSAPS |
| CCM104A | Met104→Ala | 10 | MKSSEPASVSAAERRAETFQHKLEQFNPGIV |
| HBD | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GCPVKSPPPVAAMINIPDRILMIKVSSMIAAG |
| | | | GASGTSMIFYDVTDLTTEPSGLPAGGSAPS |
| CCM104H | Met104→His | 11 | MKSSEPASVSAAERRAETFQHKLEQFNPGIV |
| HBD | Cys127→Ser | | WLDQHGRVTAFNDVALQILGPAGEQSLGVA |
| | Cys130→Ser | | QDSLFGIDVVQLHPEKSRDKLRFLLQSKDVG |
| | | | GCPVKSPPPVAHMINIPDRILMIKVSSMIAAG |
| | | | GASGTSMIFYDVTDLTTEPSGLPAGGSAPS |
| CCC Ml04A | Met104→Ala | 12 | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVAAMINIPDRILMIKVSSMIAAG |
| | | | GASGTSMIFYDVTDLTTEPSGLPAGGSAPS |
| CCC M104H | Met104→His | B | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVAHMINIPDRILMIKVSSMIAAG |
| | | | GASGTSMIFYDVTDLTTEPSGLPAGGSAPS |
| CCC M104L | Met104→Leu | 14 | MKSSEPASVSAAERRAETFQHKLEQFNPGIV |
| HBD | Cys94→Ser | | WLDQHGRVTAFNDVALQILGPAGEQSLGVA |
| | Cys127→Ser | | QDSLFGIDVVQLHPEKSRDKLRFLLQSKDVG |
| | Cys130→Ser | | GSPVKSPPPVALMINIPDRILMIKVSSMIAAG |
| | | | GASGTSMIFYDVTDLTTEPSGLPAGGSAPS |

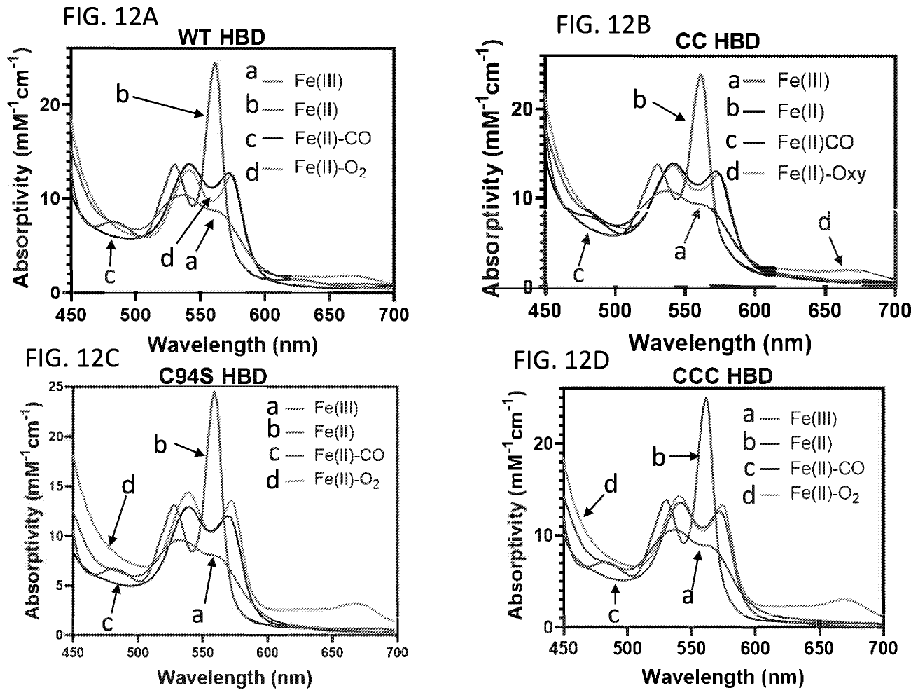

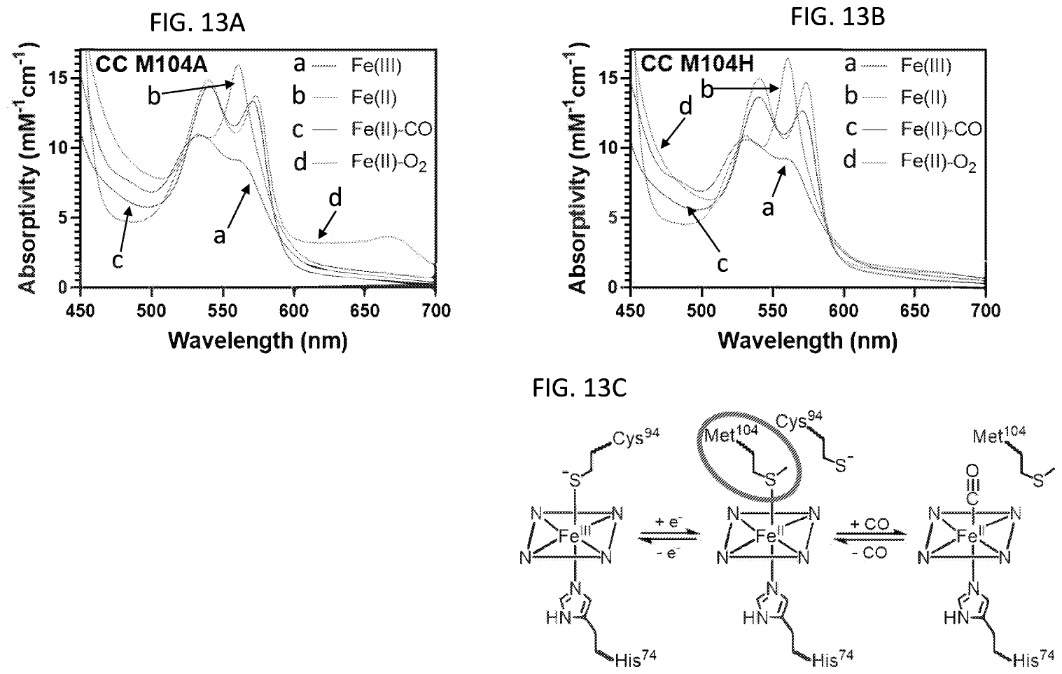

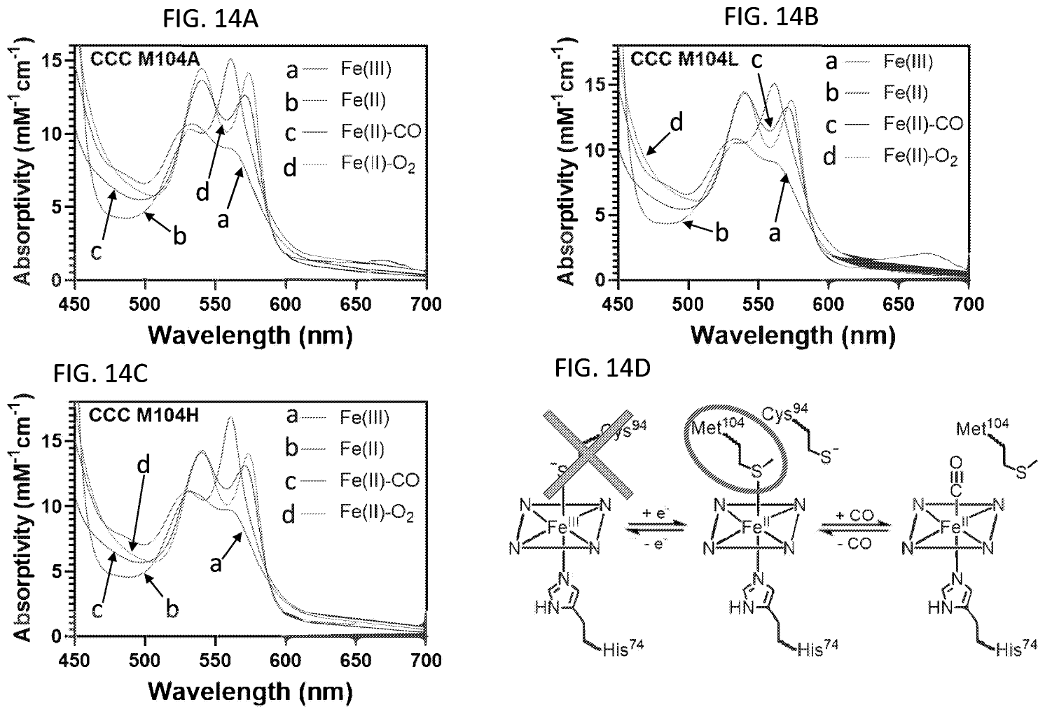

Electronic absorption (UV-Vis) spectra for RcoM HBD WT and variants are shown in FIGS. 12A-12D, 13A-13B and 14A-14C. A schematic for the protein-derived ligand switching mechanism for RcoM that highlights coordination sphere changes in the M104 variants is shown in FIG. 13C. A schematic for the protein-derived ligand switching mechanism for RcoM that highlights coordination sphere changes for the CCC M104 variants is shown in FIG. 14D.

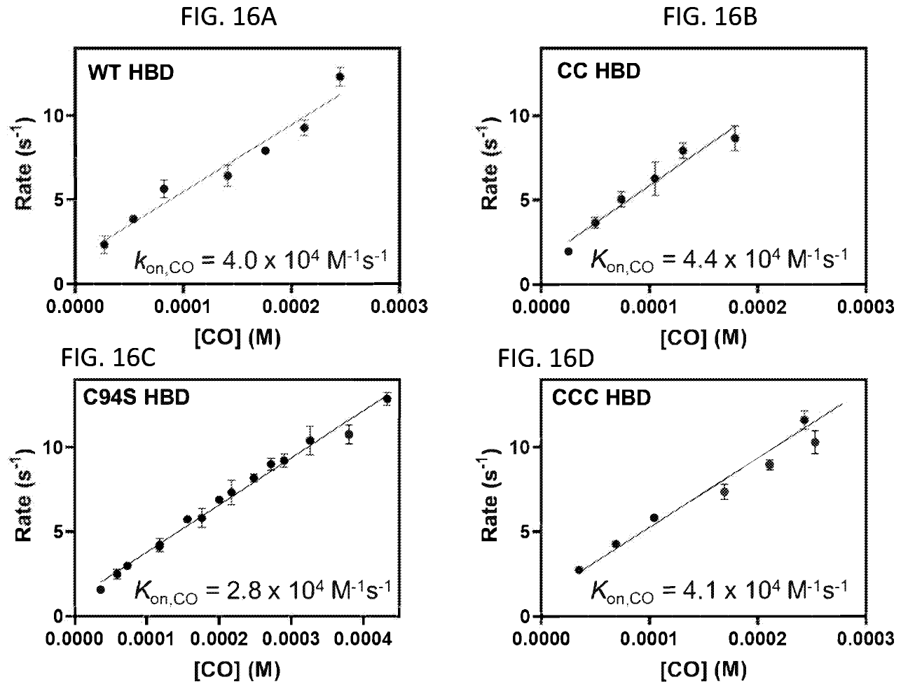

Quantification of oxygen binding affinity (P₅₀) in RcoM HBD truncates is shown in FIGS. 15A-15D. The fraction of hemoprotein bound to oxygen was measured as a function of oxygen partial pressure using UV-Vis spectroscopy using a tonometer apparatus equipped with an optical cuvette. Representative spectral changes in UV-Vis features for CC HBD RcoM variant as a function of oxygen partial pressure (P_{O2}) is shown in FIG. 15A. Oxygen binding curves for CC HBD, C94S HBD and CCC HBD are shown in FIGS. 15B-15D. Second order rate constants for CO binding (k_{on,CO}) to RcoM WT HBD and HBD variants CC HBD, C94S HBD and CCC HBD were determined (FIGS. 16A-16D). The CO binding rate at each concentration of CO was measured using stopped-flow UV-Vis spectroscopy and fit to a single exponential. A linear regression was applied to each curve, and the second order rate constant was estimated as the slope. The results were as follows:

| RcoM Variant | k_{on, CO} |
|---|---|
| WT HBD | 4.0 × 10⁴ M⁻¹s⁻¹ |
| CC HBD | 4.4 × 10⁴ M⁻¹s⁻¹ |
| C94S HBD | 2.8 × 10⁴ M⁻¹s⁻¹ |
| CCC HBD | 4.1 × 10⁴ M⁻¹s⁻¹ |

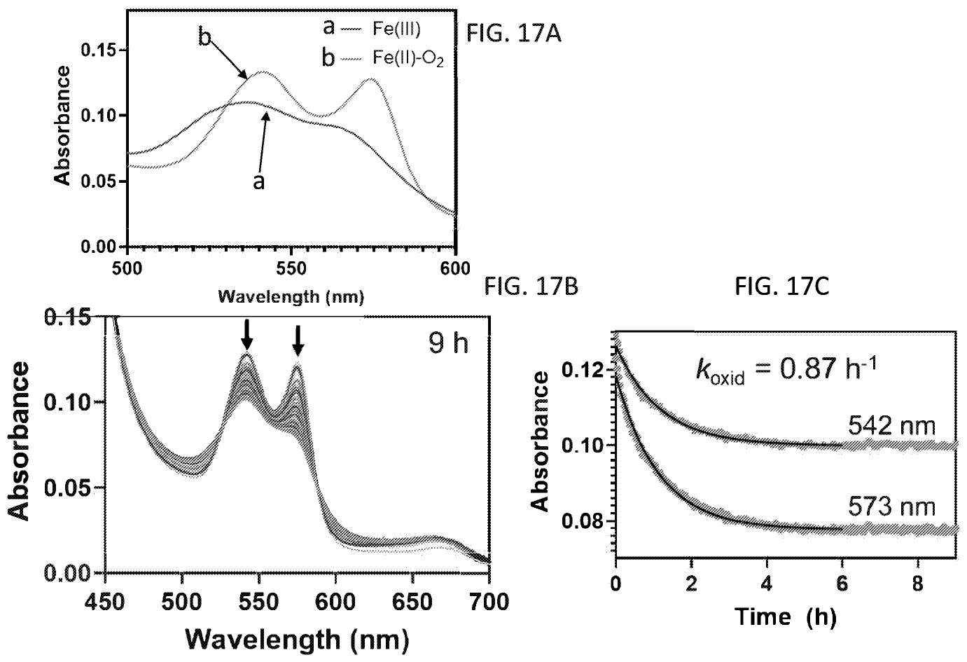

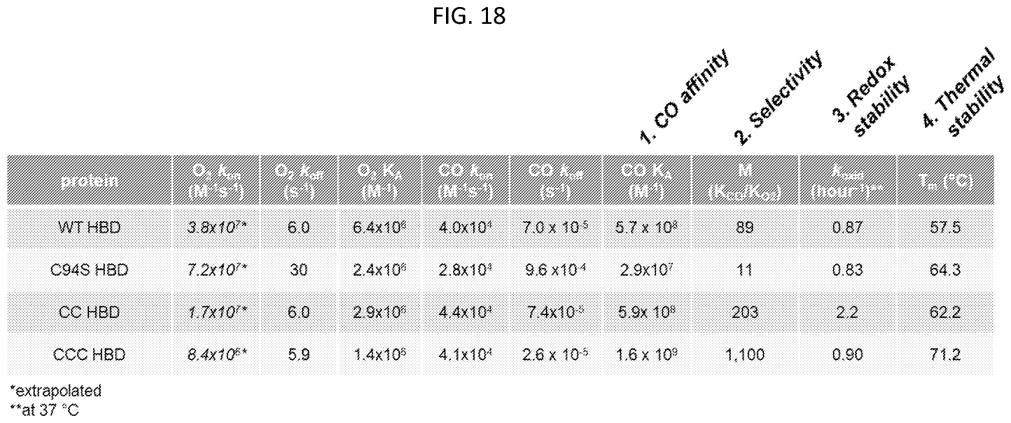

The autooxidation rate (k_{oxid}) for WT HBD RcoM was determined to be 0.87 h⁻¹. FIG. 17A shows reference spectra for Fe(III) and Fe(II)-O₂ proteins. Spectral changes in UV-Vis features for Fe(II)-O₂ WT HBD is shown in FIG. 17B. Spectral changes at 542 nm and 573 nm were fit to a single exponential to determine k_{oxid} (FIG. 17C). FIG. 18 shows a table providing a summary of ligand binding parameters and heme stability properties for WT RcoM and RcoM HBD variants C94S, CC HBD and CCC HBD.

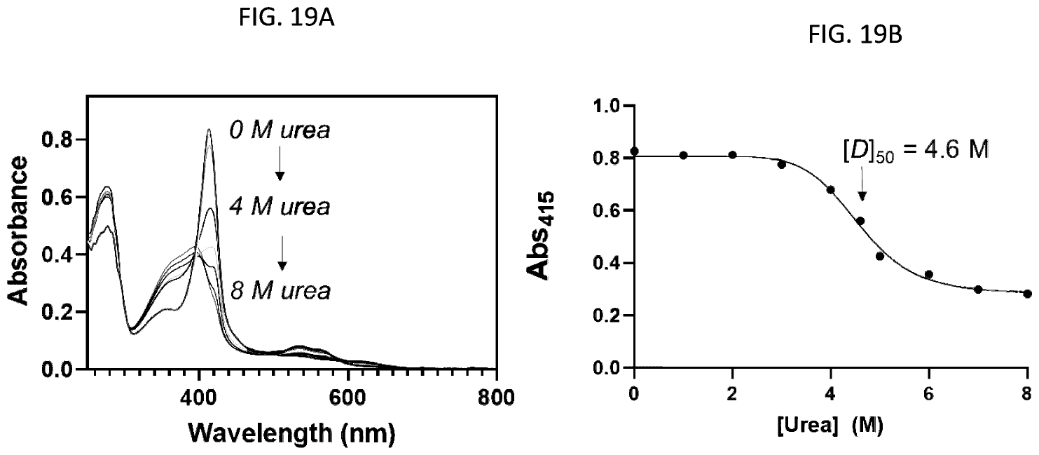

Unfolding of Fe(III) CCC HBD RcoM in the presence of urea (0 M, 4 M and 8M urea) was evaluated. Unfolding was monitored by changes in absorbance at the heme Soret maximum at 415 nm. Samples were allowed to equilibrate for 10 minutes before recording each UV-Vis spectrum (FIG. 19A). Unfolding data were fit to a sigmoidal curve to determine the concentration of denaturant at which half of the protein sample was unfolded ([D]₅₀) (FIG. 19B). [D]₅₀ for CCC HBD was 4.6 M.

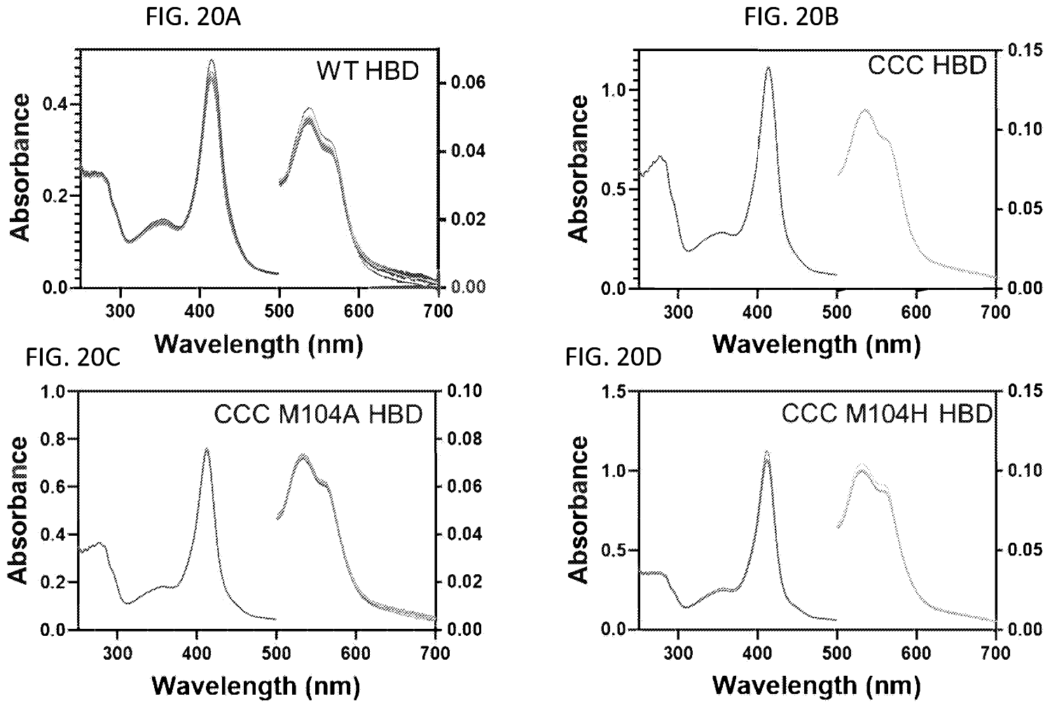

Reactivity between RcoM HBD variants and hydrogen peroxide was also evaluated. Fe(III) WT HBD and variants CCC HBD, CCC M104A HBD and CCC M104H HBD were incubated with 500 μM hydrogen peroxide at pH 7.4, 25° C. and monitored by UV-Vis spectroscopy every 2 minutes over the course of 30 minutes (FIGS. 20A-20D). Minimal spectral changes were observed for each variant, suggesting that hydrogen peroxide does not react with the Fe(III) heme center of RcoM HBD truncates to produce highly oxidizing species.

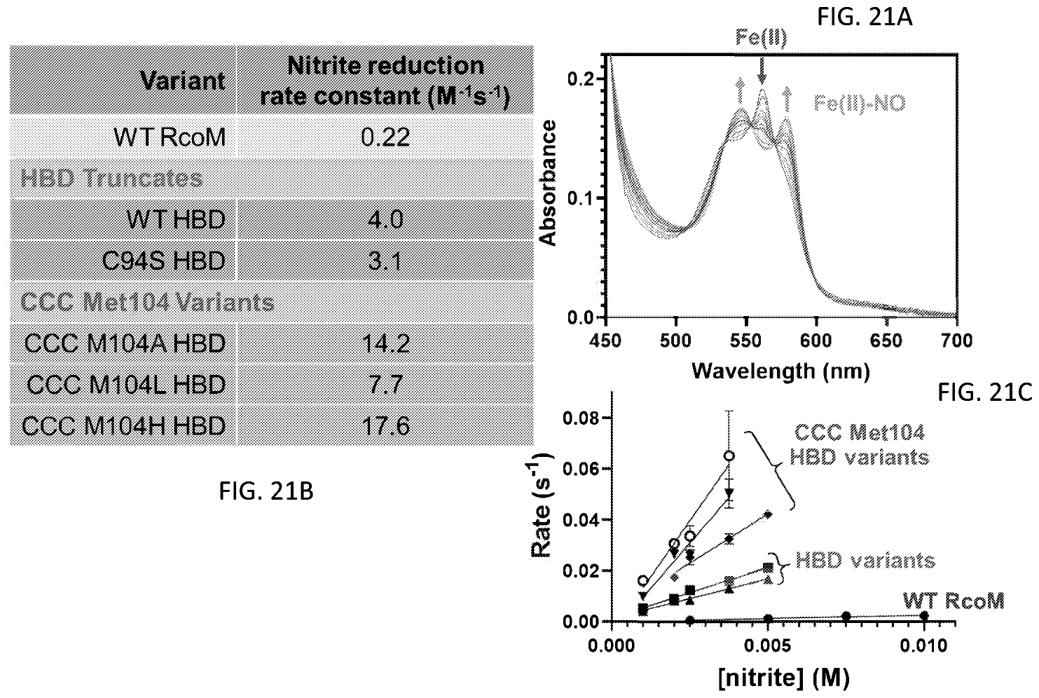

Nitrite reduction for full-length and HBD truncate RcoM variants was assessed. Ferrous protein (10-15 μM) was incubated with 1-5 mM sodium nitrite at 37° C. in the presence of 2.5 mM sodium dithionite. UV-Vis spectroscopy was used to monitor the conversion of Fe(II) heme to Fe(II)-NO (FIG. 21A). Changes in spectral features at 562 nm and 578 nm were fit to a single exponential curve to determine observed rates of nitrite reduction. Observed rates were plotted as a function of nitrite concentration, a linear regression was applied to each plot with the second order rate constant estimated as the slope (FIGS. 21B-21C).

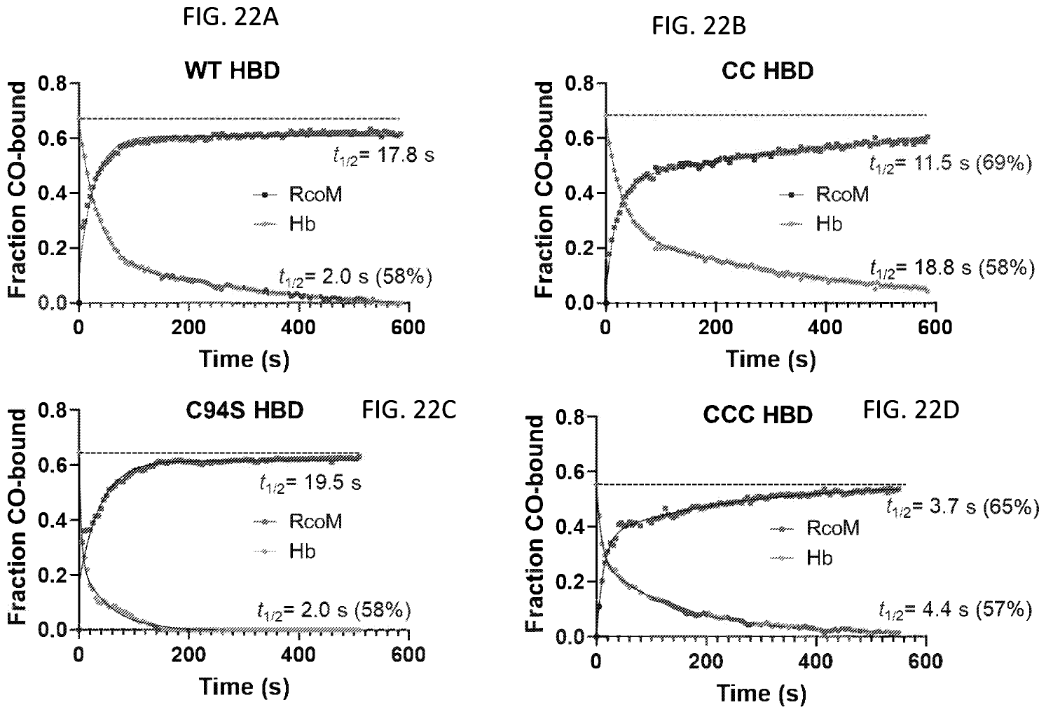

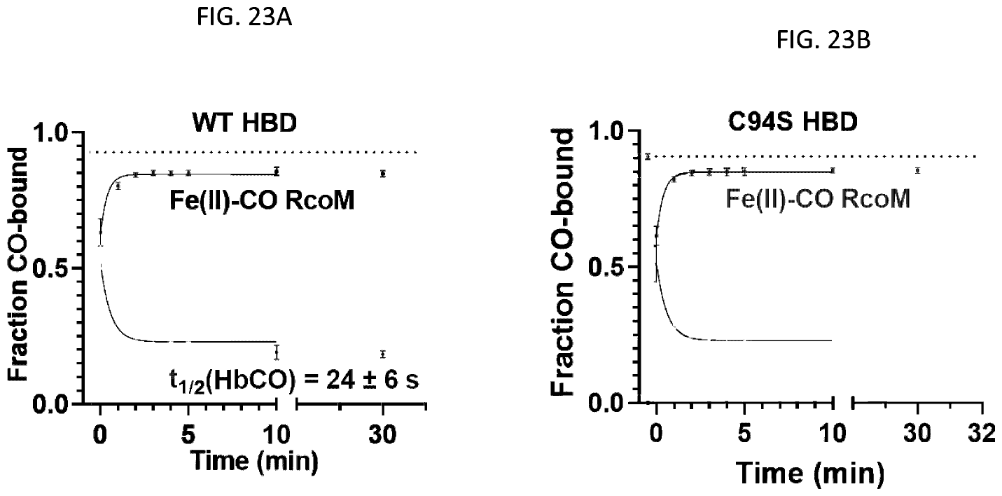

Additional studies were performed to assess the CO scavenging ability of RcoM. Kinetic traces were developed for in vitro CO transfer from hemoglobin (Hb) to WT RcoM HBD and RcoM HBD variants CC HBD, C94S HBD and CCC HBD under aerobic conditions at 37° C. CO-bound Hb (20 μM) was incubated with equimolar oxyferrous RcoM, and CO transfer from Hb to RcoM was monitored using UV-Vis spectroscopy. The fraction of each CO-bound hemoprotein was determined using spectral deconvolution, and corresponding kinetic traces were fit to a single or double exponential equation. The half-life of each CO-bound species is displayed in FIGS. 22A-22D, with the fast species half-life and amplitude displayed for curves fit to double exponentials. FIGS. 23A-23B show kinetic traces monitoring CO transfer from red blood cell (RBC)-encapsulated HbCO to extracellular RcoM HBD truncates under aerobic conditions at 37° C. Hemoproteins were incubated at equimolar concentrations (50-100 μM), and RBCs were separated from extracellular RcoM by centrifugation at each time point. CO transfer from Hb to WT HBD RcoM (FIG. 23A) and C94S HBD RcoM (FIG. 23B) was monitored using UV-Vis spectroscopy. The fraction of each CO-bound hemoprotein was determined using spectral deconvolution, and corresponding kinetic traces were fit to a single exponential equation. The half-life of COHb in the presence of WT HBD and C94S HBD was 24±6 seconds and 23±5 seconds, respectively.

These results demonstrate that RcoM HBD variants rapidly scavenge CO from RBC-encapsulated Hb under aerobic conditions similar to those likely to occur in vivo during acute CO poisoning. RcoM HBD variants are selective for CO over oxygen, as CO transfer from HbCO proceeds under aerobic conditions.

### Example 5: Toxicity Screen of RcoM HBD Variants in Mice

Recombinantly expressed RcoM truncates were introduced to healthy mice via tail vein catheter at a concentration of 1 mM or 10 mM and an injection volume of 10 μL/g body weight. Behavior (including nesting) was monitored over a 48-hour period, followed by sacrifice and collection of blood for toxicological assessment. The results are shown in Table 5. Blood chemistry results indicative of liver function (AST and ALT) and kidney function (BUN and creatinine) for all mice treated with RcoM truncates were comparable to results for control mice given phosphate buffered saline (PBS). These results indicate that intravenous infusion of RcoM truncates did not elicit organ-specific toxicity in mice.

TABLE 5
Results of toxicity screen

| | | | | | | | Creat- |
|---|---|---|---|---|---|---|---|
| Protein | | Nest- | Be- | AST | ALT | BUN | inine |
| (dose) | N= | ing | havior | (U/L) | (U/L) | (mg/dL) | (mg/dL) |
| CCC HBD | 3 | 24 h | normal | 75, | 14, | 29, | 0.1, |
| (1 mM) | | | | 43, 38 | 11, 13 | 31, 28 | 0.1, 0.0 |
| CCC HBD | 1 | 48 h | normal | 56 | 17 | 23 | 0.1 |
| (10 mM) | | | | | | | |
| CCC M104A | 2 | 24 h | normal | 55, 42 | 11, 11 | 18, 27 | 0.1, 0.1 |
| HBD | | | | | | | |
| (1 mM) | | | | | | | |
| CCC M104H | 2 | 24 h | normal | 45, 43 | 10, 10 | 19, 21 | 0.1, 0.1 |
| HBD | | | | | | | |
| (1 mM) | | | | | | | |
| CCC M104L | 1 | 24 h | normal | 66 | 7 | 32 | 0.2 |
| HBD | | | | | | | |
| (1 mM) | | | | | | | |
| PBS | 1 | 24 h | normal | 28 | 13 | 30 | 0.1 |

### Example 6: RcoM CO Scavenging In Vivo

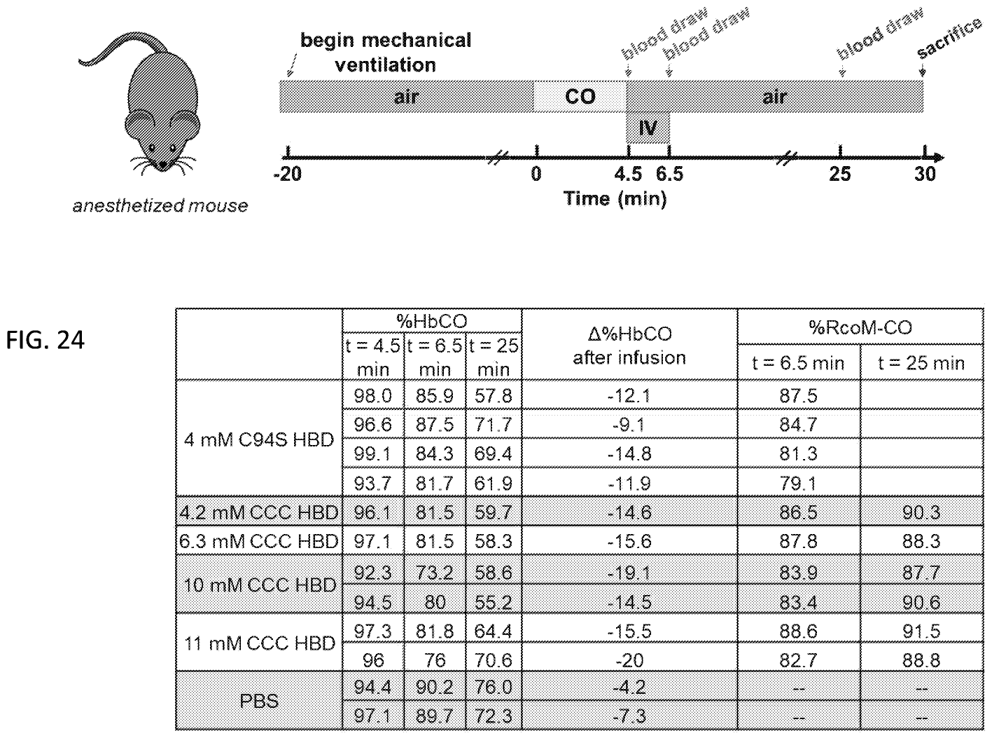

The ability of C94S and CCC HBD RcoM variants to scavenge CO from HbCO was evaluated in a lethal CO poisoning mouse model. Anesthetized, mechanically ventilated mice were exposed to 3,000 ppm CO in air for 4.5 minutes, followed by intravenous infusion of Fe(II)-O₂ CCC HBD RcoM at an injection volume of 10 μL/g body weight (hemoprotein concentrations shown in FIG. 24). Blood samples (15 μL) were drawn immediately before and after infusion, as well as 25 minutes after CO exposure. At each time point, RBCs were separated from plasma by centrifugation, and separated RBC pellets and plasma samples were immediately frozen at −80° C. Subsequently, the fraction of CO-bound hemoglobin from RBCs (% HbCO) and the fraction of CO-bound RcoM (% RcoM-CO) were determined using spectral deconvolution. Infusion with RcoM resulted in a greater decrease in the fraction of CO-bound Hb (Δ% HbCO) compared to infusion with PBS (FIG. 24), indicating that RcoM is capable of scavenging CO in vivo.

In view of the many possible embodiments to which the principles of the disclosed subject matter may be applied, it should be recognized that the illustrated embodiments are only examples of the disclosure and should not be taken as limiting the scope of the disclosure. Rather, the scope of the disclosure is defined by the following claims. We therefore claim all that comes within the scope and spirit of these claims.

1. A recombinant regulator of carbon monoxide metabolism (RcoM) protein, wherein the recombinant RcoM protein comprises a heme-binding domain (HBD), and wherein the amino acid sequence of the HBD is at least 90% identical to SEQ ID NO: 2 and comprises an amino acid substitution at one or more of H74, C94, M104, M105, C127 and C130.

2. The recombinant RcoM protein of claim 1, wherein:
the substitution at H74 is selected from H74S, H74T, H74M, H74W, H74A, H74L, H74I, H74V and H74G;
the substitution at C94 is selected from C94S, C94T, C94H, C94W, C94M, C94A, C94L, C94I, C94V and C94G;
the substitution at M104 is selected from M104S, M104T, M104H, M104W, M104A, M104L, M104I, M104V and M104G;
the substitution at M105 is selected from M105S, M105T, M105H, M105W, M105A, M105L, M105I, M105V and M105G;
the substitution at C127 is selected from C127S, C127T, C127M, C127A, C127L, C127I, C127V and C127G; and/or
the substitution at C130 is selected from C130S, C130T, C130M, C130A, C130L, C130I, C130V and C130G.

3. The recombinant RcoM protein of claim 1, wherein the amino acid sequence of the HBD is at least 95% identical to SEQ ID NO: 2 and comprises an amino acid substitution at one or more of C94, M104, C127 and C130.

4. The recombinant RcoM protein of claim 1, wherein the HBD comprises:
a C94S substitution;
a C127S substitution and a C130S substitution;
a C94S substitution, a C127S substitution and a C130S substitution;
a M104A substitution, a C127S substitution and a C130S substitution;
a M104H substitution, a C127S substitution and a C130S substitution;
a M104L substitution, a C127S substitution and a C130S substitution;
a C94S substitution, a M104A substitution, a C127S substitution and a C130S substitution;
a C94S substitution, a M104H substitution, a C127S substitution and a C130S substitution; or
a C94S substitution, a M104L substitution, a C127S substitution and a C130S substitution.

5. The recombinant RcoM protein of claim 1, wherein:
the amino acid sequence of the RcoM protein comprises or consists of SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14; or
the amino acid sequence of the RcoM protein comprises or consists of SEQ ID NO: 1 or SEQ ID NO: 2, except for an amino acid substitution at one or more of H74, C94, M104, C127, C130 and M105.

6. The recombinant RcoM protein of claim 1, wherein the RcoM protein comprises an N-terminal tag or a C-terminal tag.

7-8. (canceled)

9. The recombinant RcoM protein of claim 6, wherein the tag is cleavable.

10. An in vitro method of removing carbon monoxide from hemoglobin, myoglobin or mitochondria in blood or animal tissue, comprising contacting the blood or animal tissue with an effective amount of the recombinant RcoM protein of claim 1, thereby removing carbon monoxide from hemoglobin in the blood or animal tissue.

11. A method of treating carboxyhemoglobinemia in a subject, comprising administering to the subject a therapeutically effective amount of the RcoM protein of claim 1.

12. The method of claim 11, further comprising selecting a subject with carboxyhemoglobinemia prior to administering the recombinant RcoM protein.

13. The method of claim 11, wherein the subject has at least 5%, at least 10%, at least 15%, at least 20%, at least 30%, at least 40% or at least 50% carboxyhemoglobin in their blood.

14. The method of claim 11, wherein:
the recombinant RcoM protein is administered by intravenous infusion, intraperitoneal injection or intramuscular injection;
the recombinant RcoM protein is administered at a dose of about 0.1 g to about 300 g per day; and/or
the recombinant RcoM protein is administered as a pharmaceutical composition comprising a reducing agent.

15-17. (canceled)

18. A method of treating cyanide poisoning in a subject, comprising administering to the subject a therapeutically effective amount of the recombinant RcoM protein of claim 1, wherein the RcoM protein is in its oxidized form, thereby treating cyanide poisoning in the subject.

19. The method of claim 18, further comprising selecting a subject with cyanide poisoning prior to administering the recombinant RcoM protein.

20. The method of claim 18, wherein the recombinant RcoM protein is administered as a pharmaceutical composition comprising an oxidizing agent.

21. (canceled)

22. A method of treating hydrogen sulfide (H₂S) poisoning in a subject, comprising administering to the subject a therapeutically effective amount of the recombinant RcoM protein of claim 1, wherein the RcoM protein is in its reduced form, thereby treating H₂S poisoning in the subject.

23. The method of claim 22, further comprising selecting a subject with H₂S poisoning prior to administering the recombinant RcoM protein.

24. The method of claim 22, wherein the recombinant RcoM protein is administered as a pharmaceutical composition comprising a reducing agent.

25. (canceled)

26. A method of replacing blood in a subject, comprising administering to the subject a therapeutically effective amount of the recombinant RcoM protein of claim 1, thereby replacing blood in the subject.

27. The method of claim 26, wherein:
the subject has or is at risk of developing a disease, disorder or injury associated with a deficiency in red blood cells and/or hemoglobin, or associated with a reduction in oxygen delivery to tissues; or
the subject suffers from or is at risk of suffering from myocardial infarction, stroke, ischemia-reperfusion injury, pulmonary hypertension or vasospasm.

28-30. (canceled)

31. The method of claim 26, wherein the recombinant RcoM protein is administered to the subject intravenously.

32. The method of claim 26, wherein the recombinant RcoM protein is pegylated, polymerized or cross-linked.

33. The method of claim 26, further comprising administering to the subject a second blood replacement product, a blood product or whole blood.

34-37. (canceled)

38. A pharmaceutical composition, comprising the recombinant RcoM protein of claim 1 and a pharmaceutically acceptable carrier.

39. The pharmaceutical composition of claim 37, further comprising a reducing agent or an oxidizing agent.

40. The pharmaceutical composition of claim 39, wherein:
the reducing agent comprises sodium dithionite, ascorbic acid, N-acetylcysteine (NAC), methylene blue, glutathione, cytochrome b5/b5-reductase, hydralazine, tris(2-carboxyethyl)phosphine (TCEP), dithiothreitol (DTT), or any combination thereof; or
the oxidizing agent comprises an oxygen-containing gas mixture, an oxygen-containing liquid mixture, a ferricyanide salt, a quinone, or any combination thereof.

41. (canceled)

42. The pharmaceutical composition of claim 38, wherein the recombinant RcoM protein is pegylated, polymerized or cross-linked.


SEQUENCE LISTING (14 sequences)

SEQ ID NO: 1 - protein, 267 aa, Paraburkholderia xenovorans
MKSSEPASVSAAERRAETFQHKLEQFNPGIVWLDQHGRVTAFNDVALQILGPAGEQSLGVAQDSLFGIDVVQLHPEKSRDKLRFLLQSKDVGGCPVKSPPPVAMMINIPDRILMIKVSSMIAAGGACGTCMIFYDVTDLTTEPSGLPAGGSAPSPRRLFKIPVYRKNRVILLDLKDIVRFQGDGHYTTIVTRDDRYLSNLSLADLELRLDSSIYLRVHRSHIVSLQYAVELVKLDESVNLVMDDAEQTQVPVSRSRTAQLKELLGVV

SEQ ID NO: 2 - protein, 154 aa, Paraburkholderia xenovorans
MKSSEPASVSAAERRAETFQHKLEQFNPGIVWLDQHGRVTAFNDVALQILGPAGEQSLGVAQDSLFGIDVVQLHPEKSRDKLRFLLQSKDVGGCPVKSPPPVAMMINIPDRILMIKVSSMIAAGGACGTCMIFYDVTDLTTEPSGLPAGGSAPS

SEQ ID NO: 3 - protein, 108 aa, Paraburkholderia xenovorans
NPGIVWLDQHGRVTAFNDVALQILGPAGEQSLGVAQDSLFGIDVVQLHPEKSRDKLRFLLQSKDVGGCPVKSPPPVAMMINIPDRILMIKVSSMIAAGGACGTCMIFY

SEQ ID NO: 4 - protein, 250 aa, Hydrogenophaga crassostreae
MEAEVANKSPLYLLEKFEVGVIHLDAKRTVLAMNDFARKVLPVGEKQPFDKLVSSFHPARSKPKVDFLLDQASSCPMVSAVPMTMIINIPEQVLLIKVTRLADHMGKTTGFVLVFYDVTQVVSQEVAASEPPSTSVRLTRIPMVANHKVAFVDTQDVLCLESQAHSTRILTRDGFHFCNLSIGDLESRLDPEQFMRIHRCFIVNLQGVAELGREGSKTHVVLKGKNKEPVPVARGDVLRLRKALGLLSRH

SEQ ID NO: 5 - protein, 7 aa, Artificial Sequence
  Synthetic peptide
  MISC_FEATURE (7)..(7): Xaa = Gly or Ser
ENLYFQX

SEQ ID NO: 6 - protein, 6 aa, Artificial Sequence
  Synthetic peptide
LVPRGS

SEQ ID NO: 7 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGSPVKSPPPVAMMINIPDRILMIKVSSMIAAGGACGTCMIFYDVTDLTTEPSGLPAGGSAPS

SEQ ID NO: 8 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGCPVKSPPPVAMMINIPDRILMIKVSSMIAAGGASGTSMIFYDVTDLTTEPSGLPAGGSAPS

SEQ ID NO: 9 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGSPVKSPPPVAMMINIPDRILMIKVSSMIAAGGASGTSMIFYDVTDLTTEPSGLPAGGSAPS

SEQ ID NO: 10 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGCPVKSPPPVAAMINIPDRILMIKVSSMIAAGGASGTSMIFYDVTDLTTEPSGLPAGGSAPS

SEQ ID NO: 11 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGCPVKSPPPVAHMINIPDRILMIKVSSMIAAGGASGTSMIFYDVTDLTTEPSGLPAGGSAPS

SEQ ID NO: 12 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGSPVKSPPPVAAMINIPDRILMIKVSSMIAAGGASGTSMIFYDVTDLTTEPSGLPAGGSAPS

SEQ ID NO: 13 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGSPVKSPPPVAHMINIPDRILMIKVSSMIAAGGASGTSMIFYDVTDLTTEPSGLPAGGSAPS

SEQ ID NO: 14 - protein, 154 aa, Artificial Sequence
  Synthetic polypeptide
MKSSEPASVSAAERRAETFQHKLEQFNPGIVWLDQHGRVTAFNDVALQILGPAGEQSLGVAQDSLFGIDVVQLHPEKSRDKLRFLLQSKDVGGSPVKSPPPVALMINIPDRILMIKVSSMIAAGGASGTSMIFYDVTDLTTEPSGLPAGGSAPS